(19) United States
(12) Patent Application Publication
(10) Pub. No.: US 2002/0098159 A1
(43) Pub. Date: Jul. 25, 2002

(54) Antimicrobial compositions

(76) Inventors: Karl Shiqing Wei, Mason, OH (US); Kimberly Ann Biedermann, Cincinnati, OH (US); Jeffrey Michael Morgan, Springboro, OH (US); Peter William Beerse, Morrow, OH (US); David Edmund Tarantino, Loveland, OH (US); Mark Richard Sine, Windsor (GB); Raphael Warren, Cincinnati, OH (US)

Correspondence Address: William J. Winter, The Procter & Gamble Company, Sharon Woods Technical Center, 11511 Reed Hartman Highway, Cincinnati, OH, 45241, US

(21) Appl. No.: 09/861,132

(22) Filed: May 18, 2001

Related U.S. Application Data
(63) Continuation of application No. 09/603,948, filed on Jun. 27, 2000.
(60) Provisional application No. 60/191,939, filed on Mar. 24, 2000.
(60) Provisional application No. 60/177,092, filed on Jan. 20, 2000.
(60) Provisional application No. 60/177,091, filed on Jan. 20, 2000.

(51) Int. Cl. A61K 7/06

(52) U.S. Cl. 424/70.1

(57) ABSTRACT

The present invention relates to antimicrobial compositions comprising surfactants and organic and/or inorganic acids wherein the compositions and/or the surfactant and acid combination meet specific functional criteria. Articles of manufacture and methods of cleansing the skin using the described compositions are also disclosed.

### CROSS REFERENCE

This application claims priority under Title 35, United States Code 119(e) from application Ser. No. 09/603,948, filed Jun. 27, 2000, Provisional Application Serial No. 60/191,939, filed Mar. 24, 2000, Provisional Application Serial No. 60/177,092, filed Jan. 20, 2000, and Provisional Application Serial No. 60/177,091, filed Jan. 20, 2000.

### TECHNICAL FIELD

The present invention relates to antimicrobial compositions comprising surfactants and organic and/or inorganic acids wherein the compositions and/or the surfactant and acid combination meet specific functional criteria. Articles of manufacture and methods of cleansing the skin using the described compositions are also disclosed.

### BACKGROUND OF THE INVENTION

Human health is impacted by a variety of microbial organisms. Inoculation of humans or other mammals by these microorganisms often results in various sicknesses and ailments. Public awareness of such contaminations has been heightened due to the increased number of food poisonings, streptococcal infections, etc. which have been occurring in the recent past. Consequently, there has been a thrust by the medical community to persuade the general public to wash any areas which generally come in contact with infected surfaces like body parts (e.g. hand washing), foods (e.g., uncooked meat, vegetables, fruits, etc.), cooking utensils, cooking surfaces (e.g., counter tops, sinks, etc.). It has been found that such methods are important in attempts to remove pathogenic microorganisms from human skin as well as other surfaces.

The types of microorganisms which can be found on mammalian skin include viruses, bacteria, and fungi. In general, virologists agree that rhinoviruses, influenza viruses, and adenoviruses are most likely the most relevant viruses which cause respiratory diseases. It is believed that rhinoviruses, in particular, are responsible for acting as the primary cause for the common cold. Rhinoviruses are members of the picornavirus family. As such they are referred to as “naked viruses” since they lack an outer envelope. Such picornaviruses are known to be difficult to inactivate by commonly used means like quaternary ammonium compounds.

Rhinovirus infections are spread from person to person by means of virus-contaminated respiratory secretions. Evidence suggests that the primary mode of transmission is via direct contact, as opposed to inhalation of airborne viral particles. It has been demonstrated that ill persons have a propensity to contaminate their hands and environmental objects. Rhinovirus has been recovered from 40 to 90% of hands of persons experiencing colds and from 6 to 15% of diverse objects. Rhinovirus exhibits good survival on many environmental surfaces for hours after contamination, and infection is readily transmitted by finger-to-finger contact and by finger to contaminated environmental surface if the newly contaminated finger is then used to rub an eye or touch the nasal mucosa.

Since a substantial proportion of rhinovirus colds are transmitted by direct contact from virus-contaminated hands or objects, it is possible to lower the risk of acquiring infection by inactivating virus on hands or surfaces. A common household phenol/alcohol disinfectant has been shown to be effecting in disinfecting contaminated environmental surfaces but lacks residual virucidal effects. Hand washing is highly effective at disinfecting contaminated fingers but again suffers from a lack of residual activity. These shortcomings provide strong opportunities for improved virucidal technologies with residual activity against rhinoviruses.

It has been found that iodine is an effective anti-viral agent and provides residual anti-rhinoviral activity on skin. In experimentally induced and natural cold transmission studies, subjects who used iodine products had significantly fewer colds than placebo users. This indicates that iodine is effective for prolonged periods at blocking the transmission of rhinoviral infections. Thus, the development of hand products, lotions, or washes (without the associated color or odor negatives of iodine) that deliver both immediate and residual anti-viral activity would be effective in reducing the incidents of colds. Likewise, a topical product which exhibits anti-viral activity would be effective in preventing and/or treating virus-induced diseases caused by other viruses like adenoviruses, rotaviruses, herpes viruses, respiratory syncytial viruses, coronaviruses, parainfluenza viruses, enteroviruses, influenza viruses, etc.

With regard to bacteria, there are two types. Resident bacteria are Gram positive bacteria which are established as permanent microcolonies on the surface and outermost layers of mammalian skin. Such bacteria play an important role in preventing the colonization of other more harmful bacteria and fungi. Transient bacteria, however are not part of the normal resident flora of the skin but they can be deposited when airborne contaminated material lands on the skin or when contaminated material is brought into physical contact with it. Transient bacteria are typically divided into two subclasses: Gram positive and Gram negative. Gram positive bacteria include pathogens such as Staphylococcus aureus, Streptococcus pyogenes and Clostridium botulinum. Gram negative bacteria include pathogens such as Salmonella, Escherichia coli, Klebsiella, Haemophilus, Pseudomonas aeruginosa, Proteus and Shigella dysenteriae. Gram negative bacteria are generally distinguished from Gram positive by an additional protective cell membrane which generally results in the Gram negative bacteria being less susceptible to topical antibacterial actives.

As with viruses, the types of bacteria that can infect humans and other mammals are innumerable. As a result, a number of products have been developed over the years which are effective for providing immediate antimicrobial efficacy, that is, anti-viral and/or antibacterial efficacy. These products range from personal cleansing products such as hand soaps to household cleaning products like disinfectant sprays and cleansers. Most of these products, however, fail to provide residual activity or efficacy against pathogenic viruses and bacteria to the areas they are used to treat. A need, however, still remains for compositions and products which provide not only improved immediate anti-viral and/or antibacterial efficacy but improved residual efficacy and antifungal efficacy as well. There is also a need to provide improved immediate anti-viral (e.g., anti-rhinoviral) activity, and antibacterial activity in water based systems (i.e., non-alcohol). There is an additional need to provide compositions and products which exhibit improved antifungal efficacy.

Furthermore, although a number of antimicrobial cleansing products currently exist, taking on a variety product forms (e.g., deodorant soaps, hard surface cleaners, and surgical disinfectants), such antimicrobial products are typically rinse-off products incorporating, especially in the case of hard surface cleansers and surgical disinfectants, high levels of alcohol and/or harsh surfactants which have been shown to dry out and irritate skin tissues. Ideally, personal cleansing products should gently cleanse the skin, cause little or no irritation, and not leave the skin overly dry after frequent use and preferably should provide a moisturizing benefit to the skin.

Given the health impacts of bacterial and viral organisms, it would be highly desirable to formulate antimicrobial cleansing products which provides improved germ reduction on the skin, which are mild to the skin and which can be used without water. Existing products have been unable to deliver all of these benefits.

Applicants have discovered that personal cleansing products providing improved mildness and a new level of germ reduction can be formulated by using the improved antimicrobial compositions of the present invention. These compositions contain a unique combination organic and/or inorganic acids as proton donating agents, and surfactants, all of which are deposited on the skin. The deposited proton donating agent and surfactant provide a new level of hostility to bacteria and viruses contacting the skin while maintaining good mildness characteristics.

### SUMMARY OF THE INVENTION

The present invention relates to leave-on antimicrobial compositions comprising;

a) a proton donating agent; and

b) a surfactant

wherein the composition has:

i) Mildness Index of greater than about 0.3;

ii.) an Antibacterial Residual Effectiveness Index of greater than about 1.0; and

iii.) a Ten Minute Antiviral Residual Effectiveness Index of greater than about 1.0.

and wherein the pH of the composition is less than 5 and preferably wherein the proton donating agent has a sting index of less than 3.5 and preferably wherein the composition is substantially free of salicylic acid.

The present invention also relates to articles of manufacture and methods of cleansing and disinfecting the skin comprising the disclosed compositions.

### DETAILED DESCRIPTION OF THE INVENTION

The antimicrobial compositions of the present invention are highly efficacious for providing an improved germ reduction on the skin, are mild to the skin and can be used without additional available water.

The phrase “antimicrobial composition,” as used herein refers generally to compositions used to inactivate, destroy or kill microorganisms (i.e., bacteria and viruses). The phrase also refers to compositions used to treat diseases caused by or associated with these microorganisms such as minor wound infections as well as mild microbial skin infections (e.g., dandruff, crotch itch, athletes foot and the like).

The compositions of the present invention can also be useful for treatment of acne. As used herein “treating acne” means preventing, retarding and/or arresting the process of acne formation in mammalian skin.

The term “safe and effective amount” as used herein means an amount of a compound or composition sufficient to significantly induce a positive benefit, antiviral/antimicrobial benefit, including independently the benefits disclosed herein, but low enough to avoid serious side effects, i.e., to provide a reasonable benefit to risk ratio, within the scope of sound judgment of the skilled artisan.

By the term “immediate” as used herein means the compositions of the present invention inactivate and/or destroy viruses on the skin area within about 5 minutes, preferably within about 1 minute, more preferably within about 30 seconds, and even more preferably within about 20 seconds, without the need for soap and water.

All percentages and ratios used herein, unless otherwise indicated, are by weight and all measurements made are at 25° C., unless otherwise designated. The invention hereof can comprise, consist of, or consist essentially of, the essential as well as optional ingredients and components described therein.

The antimicrobial composition of the present invention comprising the following essential components. These components are selected so that the efficacy and optional mildness requirements hereinafter defined for the compositions herein are met. The selection of each component is necessarily dependent on the selection of each of the other components. For example, if a weak acid is selected as the proton donating agent, then in order to realize an efficacious composition, either a more biologically active (but possibly less mild) surfactant must be employed, and/or a high level of acid within the prescribed range must be used and/or a particularly efficacious active must be employed. Similarly, if a mild, but nonefficacious surfactant is employed, then a stronger acid and/or a high level of acid may be necessary to realize an efficacious composition. If a harsh surfactant is utilized, then a mildness agent may have to be utilized. Guidelines for the selection of the individual components are provided herein.

### Essential Components

I. Ingredients

Proton Donating Agent

The antimicrobial compositions of the present invention comprise from about 0.1% to about 20%, preferably from about 0.1% to about 10%, more preferably from about 0.5% to about 8%, and most preferably from about 1% to about 5%, based on the weight of the personal cleansing composition, of a proton donating agent. By “proton donating agent” it is meant any acid compound or mixture thereof, which results in undissociated acid on the skin after use. Proton donating agents can be organic acids, including polymeric acids, mineral acids or mixtures thereof.

Organic Acids

Proton donating agents which are organic acids which remain at least partially undissociated in the neat composition and remain so when the compositions are diluted during washing and rinsing. These organic proton donating agents can be added directly to the composition in the acid form or can be formed by adding the conjugate base of the desired acid and a sufficient amount of a separate acid strong enough to form the undissociated acid from the base.

Buffering Capacity

Preferred organic proton donating agents are selected and formulated based on their buffer capacity and pKa. Buffer capacity is defined as the amount of protons (weight %) available in the formulation at the product pH for those acid groups with pKa's less than about 6.0. Buffer capacity can be either calculated using pKa's, pH, and the concentrations of the acids and conjugate bases, ignoring any pKa greater than 6.0, or it can be determined experimentally through a simple acid-base titration using sodium hydroxide or potassium hydroxide using an endpoint of pH equals 6.0.

Preferred organic proton donating agents of the antibacterial cleansing composition herein have a buffer capacity of greater than about 0.005%, more preferably greater than about 0.01%, even more preferably greater than about 0.02%, and most preferably greater than about 0.04%.

Mineral Acids

Proton donating agents which are mineral acids will not remain undissociated in the neat composition and when the compositions are diluted during washing and rinsing. Despite this, it has been found that mineral acids can be effective proton donating agents for use herein. Without being limited by theory, it is believed that the strong mineral acid, acidify the carboxylic and phosphatidyl groups in proteins of the skin cells, thereby providing in-situ undissociated acid. These proton donating agents can only be added directly to the composition in the acid form.

pH

It is critical to achieving the benefits of the invention that the undissociated acid from the proton donating agent (deposited or formed in-situ) remain on the skin in the protonated form. Therefore, the pH of the antimicrobial compositions of the present invention must be adjusted to a sufficiently low level in order to either form or deposit substantial undissociated acid on the skin. The pH of the compositions should be adjusted and preferably buffered to range from about 2.0 to about 6.0, preferably from about 2.5 to about 5.0 and more preferably from about 2.5 to about 4.5.

A non-exclusive list of examples of organic acids which can be used as the proton donating agent are adipic acid, tartaric acid, citric acid, maleic acid, malic acid, succinic acid, glycolic acid, glutaric acid, benzoic acid, malonic acid, gluconic acid, gluconolactone (especially glucono-delta-lactone), 2-pyrrolidone-5 carboxylic acid, polyacrylic acid, polymeric acids, their salts, their isomers and mixtures thereof. A non-exclusive list of examples of mineral acid for use herein are hydrochloric, phosphoric, sulfuric and mixtures thereof.

Polymeric acids are especially preferred acids for use herein from the standpoint that they cause less stinging to the skin than other acids. As used herein, the term “polymeric acid” refers to an acid with repeating units of carboxylic acid groups joined together into one chain. Suitable polymeric acids can include homopolymers, copolymers and terpolymers, but must contain at least 30 mole % carboxylic acid groups. Specific examples of suitable polymeric acids useful herein include straight-chain poly(acrylic) acid and its copolymers, both ionic and nonionic, (e.g., maleic-acrylic, sulfonic-acrylic, and styrene-acrylic copolymers), those cross-linked polyacrylic acids having a molecular weight of less than about 250,000, preferably less than about 100,000 poly (α-hydroxy) acids, poly (methacrylic) acid, and naturally occurring polymeric acids such as carageenic acid, carboxy methyl cellulose, and alginic acid. Straight-chain poly(acrylic) acids are especially preferred for use herein.

Particularly preferred for use herein are 2-pyrrolidone-5 carboxylic acid, gluconolactone, isomers thereof, and mixtures thereof.

Surfactants

The antimicrobial compositions of the present invention comprise from about 0.05% to about 20%, more preferably from about 0.1% to about 10%, most preferably from about 0.1% to about 5%, optimally from about 0.1% to about 2%, based on the weight of the antimicrobial composition, of a surfactant. The surfactant may be selected from the group consisting of anionic surfactants, cationic surfactants, amphoteric or zwitterionic surfactants, and combinations thereof. In personal care applications, anionic surfactants are preferred.

A wide variety of anionic surfactants are potentially useful herein. Nonlimiting examples of anionic lathering surfactants include those selected from the group consisting of alkyl and alkyl ether sulfates, sulfated monoglycerides, sulfonated olefins, alkyl aryl sulfonates, primary or secondary alkane sulfonates, alkyl sulfosuccinates, acyl taurates, acyl isethionates, alkyl glycerylether sulfonate, sulfonated methyl esters, sulfonated fatty acids, alkyl phosphates, acyl glutamates, alkyl sulfoacetates, acylated peptides, alkyl ether carboxylates, acyl lactylates, anionic fluorosurfactants, and mixtures thereof. Mixtures of anionic surfactants can be used effectively in the present invention.

Anionic surfactants for use in the antimicrobial compositions suitable include alkyl and alkyl ether sulfates. These materials have the respective formulae R¹O—SO₃M and R¹(CH₂H₄O)ₓ—O—SO₃M, wherein R¹ is a saturated or unsaturated, branched or unbranched alkyl group from about 8 to about 24 carbon atoms, x is 1 to 10, and M is a water-soluble cation such as ammonium, sodium, potassium, magnesium, triethanolamine, diethanolamine and monoethanolamine. The alkyl sulfates are typically made by the sulfation of monohydric alcohols (having from about 8 to about 24 carbon atoms) using sulfur trioxide or other known sulfation technique. The alkyl ether sulfates are typically made as condensation products of ethylene oxide and monohydric alcohols (having from about 8 to about 24 carbon atoms) and then sulfated. These alcohols can be derived from fats, e.g., coconut oil or tallow, or can be synthetic. Specific examples of alkyl sulfates which may be used in the compositions are sodium, ammonium, potassium, magnesium, or TEA salts of lauryl or myristyl sulfate. Examples of alkyl ether sulfates which may be used include ammonium, sodium, magnesium, or TEA laureth-3 sulfate.

Another suitable class of anionic surfactants are the sulfated monoglycerides of the form R¹CO—O—CH₂—C(OH)H—CH₂—O—SO₃M, wherein R¹ is a saturated or unsaturated, branched or unbranched alkyl group from about 8 to about 24 carbon atoms, and M is a water-soluble cation such as ammonium, sodium, potassium, magnesium, triethanolamine, diethanolamine and monoethanolamine. These are typically made by the reaction of glycerin with fatty acids (having from about 8 to about 24 carbon atoms) to form a monoglyceride and the subsequent sulfation of this monoglyceride with sulfur trioxide. An example of a sulfated monoglyceride is sodium cocomonoglyceride sulfate.

Other suitable anionic surfactants include olefin sulfonates of the form R¹SO₃M, wherein R¹ is a mono-olefin having from about 12 to about 24 carbon atoms, and M is a water-soluble cation such as ammonium, sodium, potassium, magnesium, triethanolamine, diethanolamine and monoethanolamine. These compounds can be produced by the sulfonation of alpha olefins by means of uncomplexed sulfur trioxide, followed by neutralization of the acid reaction mixture in conditions such that any sultones which have been formed in the reaction are hydrolyzed to give the corresponding hydroxyalkanesulfonate. An example of a sulfonated olefin is sodium C₁₄-C₁₆ alpha olefin sulfonate.

Other suitable anionic surfactants are the linear alkylbenzene sulfonates of the form R¹—C₆H₄—SO₃M, wherein R¹ is a saturated or unsaturated, branched or unbranched alkyl group from about 8 to about 24 carbon atoms, and M is a water-soluble cation such as ammonium, sodium, potassium, magnesium, triethanolamine, diethanolamine and monoethanolamine. These are formed by the sulfonation of linear alkyl benzene with sulfur trioxide. An example of this anionic surfactant is sodium dodecylbenzene sulfonate.

Still other anionic surfactants suitable for this cleansing composition include the primary or secondary alkane sulfonates of the form R¹SO₃M, wherein R¹ is a saturated or unsaturated, branched or unbranched alkyl chain from about 8 to about 24 carbon atoms, and M is a water-soluble cation such as ammonium, sodium, potassium, magnesium, triethanolamine, diethanolamine and monoethanolamine. These are commonly formed by the sulfonation of paraffins using sulfur dioxide in the presence of chlorine and ultraviolet light or another known sulfonation method. The sulfonation can occur in either the secondary or primary positions of the alkyl chain. An example of an alkane sulfonate useful herein is alkali metal or ammonium C₁₃-C₁₇ paraffin sulfonates.

Still other suitable anionic surfactants are the alkyl sulfosuccinates, which include disodium N-octadecylsulfosuccinamate; diammonium lauryl sulfosuccinate; tetrasodium N-(1,2-dicarboxyethyl)-N-octadecylsulfosuccinate; diamyl ester of sodium sulfosuccinic acid; dihexyl ester of sodium sulfosuccinic acid; and dioctyl esters of sodium sulfosuccinic acid.

Also useful are taurates which are based on taurine, which is also known as 2-aminoethanesulfonic acid. Examples of taurates include N-alkyltaurines such as the one prepared by reacting dodecylamine with sodium isethionate according to the teaching of U.S. Pat. No. 2,658,072 which is incorporated herein by reference in its entirety. Other examples based on taurine include the acyl taurines formed by the reaction of n-methyl taurine with fatty acids (having from about 8 to about 24 carbon atoms).

Another class of anionic surfactants suitable for use in the cleansing composition are the acyl isethionates. The acyl isethionates typically have the formula R¹CO—O—CH₂CH₂SO₃M wherein R¹ is a saturated or unsaturated, branched or unbranched alkyl group having from about 10 to about 30 carbon atoms, and M is a cation. These are typically formed by the reaction of fatty acids (having from about 8 to about 30 carbon atoms) with an alkali metal isethionate. Nonlimiting examples of these acyl isethionates include ammonium cocoyl isethionate, sodium cocoyl isethionate, sodium lauroyl isethionate, and mixtures thereof.

Still other suitable anionic surfactants are the alkylglyceryl ether sulfonates of the form R¹—OCH₂—C(OH)H—CH₂—SO₃M, wherein R¹ is a saturated or unsaturated, branched or unbranched alkyl group from about 8 to about 24 carbon atoms, and M is a water-soluble cation such as ammonium, sodium, potassium, magnesium, triethanolamine, diethanolarnine and monoethanolamine. These can be formed by the reaction of epichlorohydrin and sodium bisulfite with fatty alcohols (having from about 8 to about 24 carbon atoms) or other known methods. One example is sodium cocoglyceryl ether sulfonate.

Other suitable anionic surfactants include the sulfonated fatty acids of the form R¹-CH(SO₄)—COOH and sulfonated methyl esters of the form R¹—CH(SO₄)—CO—O—CH₃, where R¹ is a saturated or unsaturated, branched or unbranched alkyl group from about 8 to about 24 carbon atoms. These can be formed by the sulfonation of fatty acids or alkyl methyl esters (having from about 8 to about 24 carbon atoms) with sulfur trioxide or by another known sulfonation technique. Examples include alpha sulphonated coconut fatty acid and lauryl methyl ester.

Other anionic materials include phosphates such as monoalkyl, dialkyl, and trialkylphosphate salts formed by the reaction of phosphorous pentoxide with monohydric branched or unbranched alcohols having from about 8 to about 24 carbon atoms. These could also be formed by other known phosphation methods. An example from this class of surfactants is sodium mono or dilaurylphosphate.

Other anionic materials include acyl glutamates corresponding to the formula R¹CO—N(COOH)—CH₂CH₂—CO₂M wherein R¹ is a saturated or unsaturated, branched or unbranched alkyl or alkenyl group of about 8 to about 24 carbon atoms, and M is a water-soluble cation. Nonlimiting examples of which include sodium lauroyl glutamate and sodium cocoyl glutamate.

Other anionic materials include alkyl ether carboxylates corresponding to the formula R¹—(OCH₂CH₂)ₓ—OCH₂—CO₂M wherein R¹ is a saturated or unsaturated, branched or unbranched alkyl or alkenyl group of about 8 to about 24 carbon atoms, x is 1 to 10, and M is a water-soluble cation. Nonlimiting examples of which include sodium laureth carboxylate.

Other anionic materials include acyl lactylates corresponding to the formula R¹CO—[O—CH(CH₃)—CO]ₓ—CO₂M wherein R¹ is a saturated or unsaturated, branched or unbranched alkyl or alkenyl group of about 8 to about 24 carbon atoms, x is 3, and M is a water-soluble cation. Nonlimiting examples of which include sodium cocoyl lactylate.

Other anionic materials include the carboxylates, nonlimiting examples of which include sodium lauroyl carboxylate, sodium cocoyl carboxylate, and ammonium lauroyl carboxylate. Anionic flourosurfactants can also be used.

Any counter cation, M, can be used on the anionic surfactant. Preferably the counter cation is selected from the group consisting of sodium, potassium, ammonium, monoethanolamine, diethanolamine, and triethanolamine.

Cationic surfactants are also useful herein, such as those having the formula:

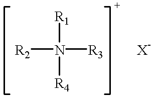

wherein R₁, is an alkyl group having from about 12 to about 30 carbon atoms, or an aromatic, aryl or alkaryl group having from about 12 to about 30 carbon atoms; R₂, R₃, and R₄ are independently selected from hydrogen, an alkyl group having from about 1 to about 22 carbon atoms, or aromatic, aryl or alkaryl groups having from about 12 to about 22 carbon atoms; and X is any compatible anion, preferably selected from the group consisting of chloride, bromide, iodide, acetate, phosphate, nitrate, sulfate, methyl sulfate, ethyl sulfate, tosylate, lactate, citrate, glycolate, and mixtures thereof. Additionally, the alkyl groups of R₁, R₂, R₃, and R₄ can also contain ester and/or ether linkages, or hydroxy or amino group substituents (e.g., the alkyl groups can contain polyethylene glycol and polypropylene glycol moieties).

More preferably, R₁ is an alkyl group having from about 12 to about 22 carbon atoms; R₂ is selected from H or an alkyl group having from about 1 to about 22 carbon atoms; R₃ and R₄ are independently selected from H or an alkyl group having from about 1 to about 3 carbon atoms; and X is as described previously.

Most preferably, R₁ is an alkyl group having from about 12 to about 22 carbon atoms; R₂, R₃, and R₄ are selected from H or an alkyl group having from about 1 to about 3 carbon atoms; and X is as described previously.

Alternatively, other useful cationic emulsifiers include amino-amides, wherein in the above structure R₁ is alternatively R₅CONH—(CH₂)ₙ, wherein R₅ is an alkyl group having from about 12 to about 22 carbon atoms, and n is an integer from about 2 to about 6, more preferably from about 2 to about 4, and most preferably from about 2 to about 3. Nonlimiting examples of these cationic emulsifiers include stearamidopropyl PG-dimonium chloride phosphate, behenamidopropyl PG dimonium chloride, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof. Especially preferred is behenamidopropyl PG dimonium chloride.

Nonlimiting examples of quaternary ammonium salt cationic surfactants include those selected from the group consisting of cetyl ammonium chloride, cetyl ammonium bromide, lauryl ammonium chloride, lauryl ammonium bromide, stearyl ammonium chloride, stearyl ammonium bromide, cetyl dimethyl ammonium chloride, cetyl dimethyl ammonium bromide, lauryl dimethyl ammonium chloride, lauryl dimethyl ammonium bromide, stearyl dimethyl ammonium chloride, stearyl dimethyl ammonium bromide, cetyl trimethyl ammonium chloride, cetyl trimethyl ammonium bromide, lauryl trimethyl ammonium chloride, lauryl trimethyl ammonium bromide, stearyl trimethyl ammonium chloride, stearyl trimethyl ammonium bromide, lauryl dimethyl ammonium chloride, stearyl dimethyl cetyl ditallow dimethyl ammonium chloride, dicetyl ammonium chloride, dicetyl ammonium bromide, dilauryl ammonium chloride, dilauryl ammonium bromide, distearyl ammonium chloride, distearyl ammonium bromide, dicetyl methyl ammonium chloride, dicetyl methyl ammonium bromide, dilauryl methyl ammonium chloride, dilauryl methyl ammonium bromide, distearyl methyl ammonium chloride, distearyl methyl ammonium bromide, and mixtures thereof. Additional quaternary ammonium salts include those wherein the C₁₂ to C₃₀ alkyl carbon chain is derived from a tallow fatty acid or from a coconut fatty acid. The term “tallow” refers to an alkyl group derived from tallow fatty acids (usually hydrogenated tallow fatty acids), which generally have mixtures of alkyl chains in the C₁₆ to C₁₈ range. The term “coconut” refers to an alkyl group derived from a coconut fatty acid, which generally have mixtures of alkyl chains in the C₁₂ to C₁₄ range. Examples of quaternary ammonium salts derived from these tallow and coconut sources include ditallow dimethyl ammonium chloride, ditallow dimethyl ammonium methyl sulfate, di(hydrogenated tallow) dimethyl ammonium chloride, di(hydrogenated tallow) dimethyl ammonium acetate, ditallow dipropyl ammonium phosphate, ditallow dimethyl ammonium nitrate, di(coconutalkyl)dimethyl ammonium chloride, di(coconutalkyl)dimethyl ammonium bromide, tallow ammonium chloride, coconut ammonium chloride, stearamidopropyl PG-dimonium chloride phosphate, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof. An example of a quaternary ammonium compound having an alkyl group with an ester linkage is ditallowyl oxyethyl dimethyl ammonium chloride.

Amphoteric and zwitterionic surfactants are also useful herein. Examples of amphoteric and zwitterionic surfactants which can be used in the compositions of the present invention are those which are broadly described as derivatives of aliphatic secondary and tertiary amines in which the aliphatic radical can be straight or branched chain and wherein one of the aliphatic substituents contains from about 8 to about 22 carbon atoms (preferably C₈-C₁₈) and one contains an anionic water solubilizing group, e.g., carboxy, sulfonate, sulfate, phosphate, or phosphonate. Examples are alkyl imino acetates, and iminodialkanoates and aminoalkanoates of the formulas RN[CH₂)ₘCO₂M]₂ and RNH(CH₂)ₘCO₂M wherein m is from 1 to 4, R is a C₈-C₂₂ alkyl or alkenyl, and M is H, alkali metal, alkaline earth metal ammonium, or alkanolammonium. Also included are imidazolinium and ammonium derivatives. Specific examples of suitable amphoteric surfactants include sodium 3-dodecyl-aminopropionate, sodium 3-dodecylaminopropane sulfonate, N-alkyltaurines such as the one prepared by reacting dodecylamine with sodium isethionate according to the teaching of U.S. Pat. No. 2,658,072 which is incorporated herein by reference in its entirety; N-higher alkyl aspartic acids such as those produced according to the teaching of U.S. Pat. No. 2,438,091 which is incorporated herein by reference in its entirety; and the products sold under the trade name “Miranol” and described in U.S. Pat. No. 2,528,378, which is incorporated herein by reference in its entirety. Other examples of useful amphoterics include phosphates, such as coamidopropyl PG-dimonium chloride phosphate (commercially available as Monaquat PTC, from Mona Corp.).

Also useful herein as amphoteric or zwitterionic surfactants are the betaines. Examples of betaines include the higher alkyl betaines, such as coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine (available as Lonzaine 16SP from Lonza Corp.), lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, coco dimethyl sulfopropyl betaine, stearyl dimethyl sulfopropyl betaine, lauryl dimethyl sulfoethyl betaine, lauryl bis-(2-hydroxyethyl) sulfopropyl betaine, and amidobetaines and amidosulfobetaines (wherein the RCONH(CH₂)₃ radical is attached to the nitrogen atom of the betaine), oleyl betaine (available as amphoteric Velvetex OLB-50 from Henkel), and cocamidopropyl betaine (available as Velvetex BK-35 and BA-35 from Henkel).

Other useful amphoteric and zwitterionic surfactants include the sultaines and hydroxysultaines such as cocamidopropyl hydroxysultaine (available as Mirataine CBS from Rhone-Poulenc), and the alkanoyl sarcosinates corresponding to the formula RCON(CH₃)CH₂CH₂CO₂M wherein R is alkyl or alkenyl of about 10 to about 20 carbon atoms, and M is a water-soluble cation such as ammonium, sodium, potassium and trialkanolamine (e.g., triethanolamine), a preferred example of which is sodium lauroyl sarcosinate.

Preferred amphoteric surfactants that are also useful herein include the amine oxides. Amine oxides are of the general form shown below, where the hydrophillic portion contains a nitrogen atom that is bound to an oxygen atom with a semipolar bond.

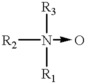

R₁,R₂,and R₃ can b a saturated or unsaturated, branched or unbranched alkyl or alkenyl group of about 1 to about 24 carbon atoms. Preferred amine oxides contain at least one R group that is an alkyl chain from 8-22 carbon atoms. Example of amine oxides include alkyl dimethyl amine oxides such as decylamine oxide (such as Barlox 10S from Lonza Inc.), cocamine oxide (such as Barlox 12 from Lonza Inc. or Mackamine Co from Macintyre Group Ltd.), myristamine oxide (such as Barlox 14 from Lonza Inc.), and palmitamineoxide (such as Barlox 16S from Lonza Inc.). Also preferred are the alkylamidopropylamineoxides, for example coamidopropylamine oxide also known as Barlox C (from Lonza Inc.).

Co-surfactants consisting of additional anionic, nonionic, cationic, and amphoteric or zwitterionic surfactants can also be included, but typically comprise less than 10% by weight of the composition.

Nonlimiting examples of preferred surfactants include those selected from the group consisting of alkyl sulfates; alkyl ether sulfates; alkyl benzene sulfonates, alpha olefin sulfonates; primary or secondary alkyl sulfonates, alkyl phosphates, alkyl sulfocarboxylates, acyl monoglyceryl sulfates; alkyl glycerylether sulfonates; acyl isethionates; acyl taurates; alkyl sulfosuccinates; alkyl sulfoacetates; sulfonated fatty acids, alkyl trimethyl ammonium chlorides and bromides, dialkyl dimethyl ammonium chlorides and bromides, alkyl dimethyl amine oxides, alkylamidopropyl amine oxides, alkyl betaines, alkyl amidopropyl betaine and mixtures thereof. More preferred surfactants include those selected from the group consisting of alkyl sulfates; alkyl ether sulfates; alkyl benzene sulfonates, alpha olefin sulfonates; primary or secondary alkyl sulfonates, alkyl phosphates, alkyl sulfocarboxylates,, alkyl trimethyl ammonium chlorides and bromides, dialkyl dimethyl ammonium chlorides and bromides, alkyl dimethyl amine oxides, alkyl betaines, and mixtures thereof. Most preferred surfactants include those selected from the group consisting of alkyl sulfates; alkyl ether sulfates; alkyl benzene sulfonates, alpha olefin sulfonates; primary or secondary alkyl sulfonates, alkyl dimethyl amine oxides, alkyl betaines and mixtures thereof.

Nonlimiting examples of preferred surfactants include those selected from the group consisting of alkyl sulfates; alkyl ether sulfates; alkyl benzene sulfonates, alpha olefin sulfonates; primary or secondary alkyl sulfonates, alkyl phosphates, alkyl sulfocarboxylates, acyl monoglyceryl sulfates; alkyl glycerylether sulfonates; acyl isethionates; acyl taurates; alkyl sulfosuccinates; alkyl sulfoacetates; sulfonated fatty acids, alkyl trimethyl ammonium chlorides and bromides, dialkyl dimethyl ammonium chlorides and bromides, alkyl dimethyl amine oxides, alkylamidopropyl amine oxides, alkyl betaines, alkyl amidopropyl betaine and mixtures thereof. More preferred surfactants include those selected from the group consisting of alkyl sulfates; alkyl ether sulfates; alkyl benzene sulfonates, alpha olefin sulfonates; primary or secondary alkyl sulfonates, alkyl phosphates, alkyl sulfocarboxylates, , alkyl trimethyl ammonium chlorides and bromides, dialkyl dimethyl ammonium chlorides and bromides, alkyl dimethyl amine oxides, alkyl betaines, and mixtures thereof. Most preferred surfactants include those selected from the group consisting of alkyl sulfates; alkyl ether sulfates; alkyl benzene sulfonates, alpha olefin sulfonates; primary or secondary alkyl sulfonates, alkyl dimethyl amine oxides, alkyl betaines and mixtures thereof.

II. Characteristics

The antimicrobial compositions herein, have the following characteristics.

Antibacterial Residual Effectiveness Index

The antimicrobial compositions of the present invention comprise an Antibacterial (or Gram negative) Residual Effectiveness Index of greater than about 1.0, preferably greater than about 1.5, more preferably greater than about 2.0, and most preferably greater than about 2.5. The Antibacterial (or Gram negative) Residual Effectiveness Index is measured by the In vitro Residual Effectiveness vs. E.coli Test described herein. The index represents a difference in base ten logarithm values of bacterial concentrations between a test sample and a placebo control. For example, an index of 0.5 represents a reduction in log values of 0.5 (Δlog=0.5) which in turn represents a 68% reduction of bacteria counts.

Antiviral Residual Efficacy Test

The antimicrobial compositions of the present invention comprise a Ten Minute Antiviral Index of greater than about 1.0, preferably greater than about 1.5, more preferably greater than about 2.0, and most preferably greater than about 2.5. The Antiviral Index is measured by the Residual Anti-viral Efficacy (or Activity) Test described herein. The index represents a difference in base ten logarithm values of viral titer concentrations between a test sample and a placebo control. For example, an index of 0.5 represents a reduction in log values of 0.5 (Δlog=0.5) which in turn represents a 68% reduction of viral titers.

Preferably, the antimicrobial compositions of the present invention comprise a One Hour Antiviral Index of greater than about 1.0, preferably greater than about 1.5, more preferably greater than about 2.0, and most preferably greater than about 2.5.

Mildness Index

The antimicrobial compositions of the present invention comprise a Mildness Index of greater than about 0.3, preferably greater than about 0.6, more preferably greater than about 1.0, most preferably greater than about 1.3, and, optimally, greater than 1.6. The Mildness Index is measured by the Forearm Controlled Application Test (FCAT) described herein.

Sting Index

The antimicrobial compositions of the present invention comprise a proton donating agent having a Sting Index of less than about 3.5, preferably less than about 3.0, more preferably less than about 2.5 and most preferably less than about 2.0. The Sting Index is measured by the Sting Test Method described herein.

### OPTIONAL COMPONENTS

Substantially Free of Salicylic Acid

Preferably the compositions of the present invention are substantially free of salicylic acid. In general, by “substantially free” what is meant is that the level of salicylic acid be up to 1.0% (or about 1.0%), more preferably up to 0.15% (or about 0.15%), most preferably up to 0.1% (or about 0.1%), and optimally zero. Without being limited by theory, salicylic acid has been reported as being irritating upon application to the skin. See, U.S. Pat. No. 4,767,750, issued Aug. 30, 1988, to Jacquet et al., herein incorporated by reference in its entirety. Accordingly, salicylic acid should not be present in the compositions of the present invention or should only be present at sufficiently low levels so as not to negatively impact the Mildness Index of the present invention.

Aqueous Component

The antimicrobial compositions described herein preferably comprise an aqueous component. For purposes of this invention the term “aqueous component” refers to any material consisting essentially of, or predominantly of water, water soluble alcohol(s) such as ethanol, propanol or isopropanol, and mixtures thereof.

The aqueous component can optionally contain one or more water-soluble emollients including, but not limited to, lower molecular weight aliphatic diols such as propylene glycol and butylene glycol; polyols such as glycerine and sorbitol; and polyoxyethylene polymers such as polyethylene glycol 200. The specific type and amount of water soluble emollient(s) employed will vary depending on the desired aesthetic characteristics of the composition, and is readily determined by one skilled in the art.

The aqueous component is preferably water which is deionized, distilled or purified. Preferred compositions comprise from about 3% to about 98.899%, preferably from about 5% to about 98%, more preferably from about 10% to about 97.5%, and most preferably from about 38% to about 95.99% of the aqueous component.

Antimicrobial Active

The antimicrobial composition of the present invention comprises from about 0.001% to about 5%, preferably from about 0.05% to about 1%, more preferably from about 0.05% to about 0.5% and more preferably from about 0.1% to about 0.25%, by weight of the antimicrobial composition, of an antimicrobial active. The exact amount of antibacterial active to be used in the compositions will depend on the particular active utilized since actives vary in potency.

Given below are examples of non-cationic antimicrobial agents which are useful in the present invention.

Pyrithiones, especially the zinc complex (ZPT)

Benzalkonium Chloride

Di(C₆-C₁₄)alkyl di short chain (C₁₋₄ alkyl and/or hydroxyalkyl)

N-(3-chloroallyl) hexaminium chlorides

Benzethonium chloride

Methylbenzethonium

Octopirox®

Dimethyldimethylol Hydantoin (Glydant®)

Methylchloroisothiazolinone/methylisothiazolinone (Kathon CG®)

Sodium Sulfite

Sodium Bisulfite

Imidazolidinyl Urea (Germall 115®) Diazolidinyl Urea (Germaill II®)

Benzyl Alcohol

2-Bromo-2-nitropropane-1,3-diol (Bronopol®)

Formalin (formaldehyde)

Iodopropenyl Butylcarbamate (Polyphase P100®)

Chloroacetamide

Methanamine

Methyldibromonitrile Glutaronitrile (1,2-Dibromo-2,4-dicyanobutane or Tektamer®)

Glutaraldehyde

5-bromo-5-nitro-1,3-dioxane (Bronidox®)

Phenethyl Alcohol

o-Phenylphenol/sodium o-phenylphenol

Sodium Hydroxymethylglycinate (Suttocide A®)

Polymethoxy Bicyclic Oxazolidine (Nuosept C®)

Dimethoxane

Thimersal

Dichlorobenzyl Alcohol

Captan

Chlorphenenesin

Dichlorophene

Chlorbutanol

Glyceryl Laurate

Halogenated Diphenyl Ethers

2,4,4′-trichloro-2′-hydroxy-diphenyl ether (Triclosan® or TCS)

2,2′-dihydroxy-5,5′-dibromo-diphenyl ether

Phenolic Compounds

Phenol

2-Methyl Phenol

3-Methyl Phenol

4-Methyl Phenol

4-Ethyl Phenol

2,4-Dimethyl Phenol

2,5-Dimethyl Phenol

3,4-Dimethyl Phenol

2,6-Dimethyl Phenol

4-n-Propyl Phenol

4-n-Butyl Phenol

4-n-Amyl Phenol

4-tert-Amyl Phenol

4-n-Hexyl Phenol

4-n-Heptyl Phenol

Mono- and Poly-Alkyl and Aromatic Halophenols

p-Chlorophenol

Methyl p-Chlorophenol

Ethyl p-Chlorophenol

n-Propyl p-Chlorophenol

n-Butyl p-Chlorophenol

n-Amyl p-Chlorophenol

sec-Amyl p-Chlorophenol

n-Hexyl p-Chlorophenol

Cyclohexyl p-Chlorophenol

n-Heptyl p-Chlorophenol

n-Octyl p-Chlorophenol

o-Chlorophenol

Methyl o-Chlorophenol

Ethyl o-Chlorophenol

n-Propyl o-Chlorophenol

n-Butyl o-Chlorophenol

n-Amyl o-Chlorophenol

tert-Amyl o-Chlorophenol

n-Hexyl o-Chlorophenol

n-Heptyl o-Chlorophenol

o-Benzyl p-Chlorophenol

o-Benxyl-m-methyl p-Chlorophenol

o-Benzyl-m, m-dimethyl p-Chlorophenol

o-Phenylethyl p-Chlorophenol

o-Phenylethyl-m-methyl p-Chlorophenol

3-Methyl p-Chlorophenol

3,5-Dimethyl p-Chlorophenol

6-Ethyl-3-methyl p-Chlorophenol

6-n-Propyl-3-methyl p-Chlorophenol

6-iso-Propyl-3-methyl p-Chlorophenol

2-Ethyl-3,5-dimethyl p-Chlorophenol

6-sec-Butyl-3-methyl p-Chlorophenol

2-iso-Propyl-3,5-dimethyl p-Chlorophenol

6-Diethylmethyl-3-methyl p-Chlorophenol

6-iso-Propyl-2-ethyl-3-methyl p-Chlorophenol

2-sec-Amyl-3,5-dimethyl p-Chlorophenol

2-Diethylmethyl-3,5-dimethyl p-Chlorophenol

6-sec-Octyl-3-methyl p-Chlorophenol

p-Chloro-m-cresol

p-Bromophenol

Methyl p-Bromophenol

Ethyl p-Bromophenol

n-Propyl p-Bromophenol

n-Butyl p-Bromophenol

n-Amyl p-Bromophenol

sec-Amyl p-Bromophenol

n-Hexyl p-Bromophenol

Cyclohexyl p-Bromophenol

o-Bromophenol

tert-Amyl o-Bromophenol

n-Hexyl o-Bromophenol

n-Propyl-m,m-Dimethyl o-Bromophenol

2-Phenyl Phenol

4-Chloro-2-methyl phenol

4-Chloro-3-methyl phenol

4-Chloro-3,5-dimethyl phenol

2,4-Dichloro-3,5-dimethylphenol

3,4,5,6-Terabromo-2-methylphenol

5-Methyl-2-pentylphenol

4-Isopropyl-3-methylphenol

Para-chloro-meta-xylenol (PCMX)

Chlorothymol

Phenoxyethanol

Phenoxyisopropanol

5-Chloro-2-hydroxydiphenylmethane

Resorcinol and its Derivatives

Resorcinol

Methyl Resorcinol

Ethyl Resorcinol

n-Propyl Resorcinol

n-Butyl Resorcinol

n-Amyl Resorcinol

n-Hexyl Resorcinol

n-Heptyl Resorcinol

n-Octyl Resorcinol

n-Nonyl Resorcinol

Phenyl Resorcinol

Benzyl Resorcinol

Phenylethyl Resorcinol

Phenylpropyl Resorcinol

p-Chlorobenzyl Resorcinol

5-Chloro 2,4-Dihydroxydiphenyl Methane

4′-Chloro 2,4-Dihydroxydiphenyl Methane

5-Bromo 2,4-Dihydroxydiphenyl Methane

4′ -Bromo 2,4-Dihydroxydiphenyl Methane

Bisphenolic Compounds

2,2′-Methylene bis (4-chlorophenol)

2,2′-Methylene bis (3,4,6-trichlorophenol)

2,2′-Methylene bis (4-chloro-6-bromophenol)

bis (2-hydroxy-3,5-dichlorophenyl) sulphide

bis (2-hydroxy-5-chlorobenzyl)sulphide

Benzoic Esters (Parabens)

Methylparaben

Propylparaben

Butylparaben

Ethylparaben

Isopropylparaben

Isobutylparaben

Benzylparaben

Sodium Methylparaben

Sodium Propylparaben

Halogenated Carbanilides

3,4,4′-Trichlorocarbanilides (Triclocarban® or TCC)

3-Trifluoromethyl-4,4′-dichlorocarbanilide

3,3′,4-Trichlorocarbanilide

A more detailed discussion of suitable antimicrobial agents can be found in U.S. Pat. No. 4,163,800; U.S. Pat. No. 3,152,181; U.S. Pat. No. 5,780,064; and Remington's pharmaceutical Sciences, 17^{th} ed. (Alfonso R. Gennaro ed., 1985) pp. 1158-1169, all of which are herein incorporated by reference in their entirety.

Another class of antibacterial agents, which are useful in the present invention, are the so-called “natural” antibacterial actives, referred to as natural essential oils. These actives derive their names from their natural occurrence in plants. Typical natural essential oil antibacterial actives include oils of anise, lemon, orange, rosemary, wintergreen, thyme, lavender, cloves, hops, tea tree, citronella, wheat, barley, lemongrass, cedar leaf, cedarwood, cinnamon, fleagrass, geranium, sandalwood, violet, cranberry, eucalyptus, vervain, peppermint, gum benzoin, basil, fennel, fir, balsam, menthol, ocmea origanum, Hydastis carradensis, Berberidaceae daceae, Ratanhiae and Curcuma longa. Also included in this class of natural essential oils are the key chemical components of the plant oils which have been found to provide the antimicrobial benefit. These chemicals include, but are not limited to anethol, catechole, camphene, carvacol, eugenol, eucalyptol, ferulic acid, farnesol, hinokitiol, tropolone, limonene, menthol, methyl salicylate, thymol, terpineol, verbenone, berberine, ratanhiae extract, caryophellene oxide, citronellic acid, curcumin, nerolidol and geraniol.

Additional active agents are antibacterial metal salts. This class generally includes salts of metals in groups 3b-7b, 8 and 3a-5a. Specifically are the salts of aluminum, zirconium, zinc, silver, gold, copper, lanthanum, tin, mercury, bismuth, selenium, strontium, scandium, yttrium, cerium, praseodymiun, neodymium, promethum, samarium, europium, gadolinium, terbium, dysprosium, holmium, erbium, thulium, ytterbium, lutetium and mixtures thereof.

Preferred antimicrobial agents for use herein are the broad spectrum actives selected from the group consisting of Benzalkonium Chloride, Benzethonium Chloride, Triclosan®, Triclocarban®, Octopirox®, PCMX, ZPT, natural essential oils and their key ingredients, and mixtures thereof. The most preferred antimicrobial active for use in the present invention is Benzalkonium Chloride®.

Mildness Enhancers

In order to achieve the mildness required of the present invention, optional ingredients to enhance the mildness to the skin can be added. These ingredients include cationic and nonionic polymers, co-surfactants, moisturizers and mixtures thereof. Polymers useful herein include polyethylene glycols, polypropylene glycols, hydrolyzed silk proteins, hydrolyzed milk proteins, hydrolyzed keratin proteins, guar hydroxypropyltrimonium chloride, polyquats, silicone polymers and mixtures thereof. When used, the mildness enhancing polymers comprise from about 0.1% to about 1%, preferably from about 0.2% to about 1.0%, and more preferably from about 0.2% to about 0.6%, by weight of the antimicrobial composition, of the composition. Co-surfactants useful herein include nonionic surfactants such as the Genapol® 24 series of ethoxylated alcohols, POE(20) sorbitan monooleate (Tween® 80), polyethylene glycol cocoate and Pluronic® propylene oxide/ethylene oxide block polymers, and amphoteric surfactants such as alkyl betaines, alkyl sultaines, alkyl amphoacetates, alkyl amphodiacetates, alkyl amphopropionates, and alkyl amphodipropionates. When used, the mildness enhancing co-surfactants comprise from about 20% to about 70%, preferably from about 20% to about 50%, by weight of the amphotericsurfactant, of the cleansing composition.

Another group of mildness enhancers are lipid skin moisturizing agents which provide a moisturizing benefit to the user of the antimicrobial compositions when the lipophilic skin moisturizing agent is deposited to the user's skin. When used in the antimicrobial compositions herein, lipophilic skin moisturizing agents are used, they are employed at a level of about 0.1% to about 30%, preferably from about 0.2% to about 10%, most preferably from about 0.5% to about 5% by weight of the composition.

In some cases, the lipophilic skin moisturizing agent can desirably be defined in terms of its solubility parameter, as defined by Vaughan in Cosmetics and Toiletries, Vol. 103, p. 47-69, October 1988. A lipophilic skin moisturizing agent having a Vaughan solubility Parameter (VSP) from 5 to 10, preferably from 5.5 to 9 is suitable for use in the antimicrobial compositions herein.

A wide variety of lipid type materials and mixtures of materials are suitable for use in the antimicrobial compositions of the present invention. Preferably, the lipophilic skin conditioning agent is selected from the group consisting of hydrocarbon oils and waxes, silicones, fatty acid derivatives, cholesterol, cholesterol derivatives, di- and tri-glycerides, vegetable oils, vegetable oil derivatives, liquid nondigestible oils such as those described in U.S. Pat. No. 3,600,186 to Mattson; Issued Aug. 17, 1971 and U.S. Pat. Nos. 4,005,195 and 4,005,196 to Jandacek et al; both issued Jan. 25, 1977, all of which are herein incorporated by reference, or blends of liquid digestible or nondigestible oils with solid polyol polyesters such as those described in U.S. Pat. No. 4,797,300 to Jandacek; issued Jan. 10, 1989; U.S. Pat. Nos. 5,306,514, 5,306,516 and 5,306,515 to Letton; all issued Apr. 26, 1994, all of which are herein incorporated by reference, and acetoglyceride esters, alkyl esters, alkenyl esters, lanolin and its derivatives, milk tri-glycerides, wax esters, beeswax derivatives, sterols, phospholipids and mixtures thereof. Fatty acids, fatty acid soaps and water soluble polyols are specifically excluded from our definition of a lipophilic skin moisturizing agent.

Hydrocarbon Oils and Waxes:

Some examples are petrolatum, mineral oil microcrystalline waxes, polyalkenes (e.g. hydrogenated and nonhydrogenated polybutene and polydecene), paraffins, cerasin, ozokerite, polyethylene and perhydrosqualene. Blends of petrolatum and hydrogenated and nonhydrogenated high molecular weight polybutenes wherein the ratio of petrolatum to polybutene ranges from about 90:10 to about 40:60 are also suitable for use as the lipid skin moisturizing agent in the compositions herein.

Silicone Oils:

Some examples are dimethicone copolyol, dimethylpolysiloxane, diethylpolysiloxane, high molecular weight dimethicone, mixed C1-C30 alkyl polysiloxane, phenyl dimethicone, dimethiconol, and mixtures thereof. More preferred are non-volatile silicones selected from dimethicone, dimethiconol, mixed C1-C30 alkyl polysiloxane, and mixtures thereof. Nonlimiting examples of silicones useful herein are described in U.S. Pat. No. 5,011,681, to Ciotti et al., issued Apr. 30, 1991, which is incorporated by reference.

Di- and Tri-glycerides:

Some examples are castor oil, soy bean oil, derivatized soybean oils such as maleated soy bean oil, safflower oil, cotton seed oil, corn oil, walnut oil, peanut oil, olive oil, cod liver oil, almond oil, avocado oil, palm oil and sesame oil, vegetable oils and vegetable oil derivatives; coconut oil and derivatized coconut oil, cottonseed oil and derivatized cottonseed oil, jojoba oil, cocoa butter, and the like.

Acetoglyceride esters are used and an example is acetylated monoglycerides.

Lanolin and its derivatives are preferred and some examples are lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate.

It is most preferred when at least 75% of the lipophilic skin conditioning agent is comprised of lipids selected from the group consisting: petrolatum, blends of petrolatum and high molecular weight polybutene, mineral oil, liquid nondigestible oils (e.g. liquid cottonseed sucrose octaesters) or blends of liquid digestible or nondigestible oils with solid polyol polyesters (e.g. sucrose octaesters prepared from C22 fatty acids) wherein the ratio of liquid digestible or nondigestible oil to solid polyol polyester ranges from about 96:4 to about 80:20, hydrogenated or nonhydrogenated polybutene, microcrystalline wax, polyalkene, paraffin, cerasin, ozokerite, polyethylene, perhydrosqualene; dimethicones, alkyl siloxane, polymethylsiloxane, methylphenylpolysiloxane and mixtures thereof. When as blend of petrolatum and other lipids is used, the ratio of petrolatum to the other selected lipids (hydrogenated or unhydrogenated polybutene or polydecene or mineral oil) is preferably from about 10:1 to about 1:2, more preferably from about 5:1 to about 1:1.

Decreasing and/or Detackifying Agent

Also essential to the compositions of the present invention are degreasing and/or detackifying agents in an effective amount to reduce the greasy feel or stickiness associated with the lipophilic skin moisturizers. The term “degreasing agent,” as used herein, means an agent which prevents, reduces and/or eliminates the greasy or heavy skin feel typically associated with lipophilic materials. The term “detackifying agent,” as used herein, means an agent which prevents, reduces and/or eliminates the sticky or tacky geeling tyupically associated with ingredients such as humectants. Degreasing or detackifying agents suitable for use in the present invention are selected from the group consisting of select silicones, wax materials soluble in the alcoholic antiseptic and having a melting point greater than about 20° C., powders, fluorochemicals and mixtures thereof.

i.) Silicones

Useful as degreasing agents in the present invention are volatile and non-volatile silicone oils. The term “nonvolatile” as used herein means that the silicone has a boiling point of at least about 260° C., preferably at least about 275° C., more preferably at least about 300° C. Such materials exhibit very low or no significant vapor pressure at ambient conditions. The term “volatile” as used herein mean that the silicone has a boiling point of from about 99° C. to about 260° C.

Volatile silicones suitable for use in the present invention are disclosed in U.S. Pat. No. 4,781,917, issued to Luebbe et al., Nov. 1, 1988 and U.S. Pat. No. 5,759,529 to LeGrow et al., issued Jun. 2, 1998, both of which are herein incorporated by reference in their entirety. Additionally, a description of various volatile silicones materials is found in Todd et al., “Volatile Silicone Fluids for Cosmetics”, Cosmetics and Toiletries, 91:27-32 (1976). Preferred silicones have surface tensions of less than about 35 dynes, more preferably less than about 30 dynes, most preferably less than about 25 dynes. Particularly preferred volatile silicone oils are selected from the group consisting of cyclic volatile silicones corresponding to the formula:

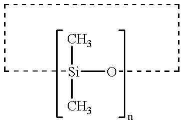

wherein n is from about 3 to about 7; and linear volatile silicones corresponding to the formula:

(CH₃)₃Si—O—[Si(CH₃)₂O].ₘ—Si(CH₃)₃

wherein m is from about 1 to about 7. Linear volatile silicones generally have a viscosity of less than about 5 centistokes at 25.degree. C., whereas the cyclic silicones have viscosities of less than about 10 centistokes at 25.degree. C. Highly preferred examples of volatile silicone oils include cyclomethicones of varying viscosities, e.g., Dow Corning 200, Dow Corning 244, Dow Corning 245, Dow Corning 344, and Dow Corning 345, (commercially available from Dow Corning Corp.); SF-1204 and SF-1202 Silicone Fluids (commercially available from G.E. Silicones), GE 7207 and 7158 (commercially available from General Electric Co.); and SWS-03314 (commercially available from SWS Silicones Corp.). When present in the compositions of the present invention, volatile silicones comprise at least about or greater than about 3% to about 10%, more preferably from about 4% to about 8%, and most preferably from about 6% to about 8% by weight of the present invention.

Also useful as the degreasing agent are nonvolatile silicones such as fluid silicones and gum silicones. The molecular weight and viscosity of the particular selected silicone will determine whether it is a gum or a fluid. The term “silicone fluid,” as used herein, denotes a silicone with viscosities ranging from about 5 to about 600,000 centistokes, most preferably from about 350 to about 100,000 centistokes, at 25° C. The term “silicone gum,” as used herein, denotes silicones with mass molecular weights of from about 200,000 to about 1,000,000 and with viscosities greater than about 600,000 centistokes. The non-volatile silicones of the present invention preferably have a viscosity of at least about 15,000 centipoise, more preferably at least 25,000 centipoise.

Suitable non-volatile silicones include polysiloxanes and other modified silicones. Polysiloxanes and other modified silicones are described in U.S. Pat. Nos. 5,650,144 and 5,840,288, both of which are herein incorporated by reference in their entirety. Examples of suitable polysiloxanes and modified silicones include, but are not limited to, polyalkylsiloxanes, polyarylsiloxanes, polyalkylarylsiloxanes, polyestersiloxanes, polyethersiloxane copolymers, polyfluorosiloxanes, polyaminosiloxanes, and mixtures thereof. Preferred non-volatile polysiloxanes are polydimethylsiloxane having viscosities of from about 5 to about 100,000 centistokes at 25° C.

Silicone fluid and gum mixtures or blends can also be used. Silicone gum and fluid blends are disclosed in U.S. Pat. No. 4,906,459, Cobb et al., issued Mar. 6, 1990; U.S. Pat. No. 4,788,006, Bolich, Jr. et al., issued Nov. 29, 1988; U.S. Pat. No. 4,741,855, Grote et al., issued May 3, 1988; U.S. Pat. No. 4,728,457, Fieler et al., issued Mar. 1, 1988; U.S. Pat. No. 4,704,272, Oh et al., issued Nov. 3, 1987; and U.S. Pat. No. 2,826,551, Geen, issued Mar. 11, 1958; U.S. Pat. No. 5,154,849, Visscher et al., issued Oct. 13, 1992, all of which are herein incorporated by reference in their entirety.

When present in the compositions of the present invention, non-volatile silicones comprise from about 0.01% to about 5%, preferably from about 0.1% to about 2%, more preferably from about 0.1% to about 1% by weight of the present invention.

Silicone elastomers are also useful as degreasing agents in the present invention. Suitable silicone elastomers are illustrated in U.S. Pat. No. 4,970,252 to Sakuta et al., issued Nov. 13, 1990; U.S. Pat. No. 5,760,116 to Kilgour et al., issued Jun. 2, 1998; U.S. Pat. No. 5,654,362 to Schulz, Jr. et al. issued Aug. 5, 1997; and Japanese Patent Application JP 61-18708, assigned to Pola Kasei Kogyo KK, as well as U.S. Pat. No. 5,412,004 (issued May 2, 1995); U.S. Pat. No. 5,837,793 (issued Nov. 17, 1998); and U.S. Pat. No. 5,811,487 (issued Sep. 22, 1998), each of which are herein incorporated by reference in its entirety. Examples of suitable elastomers include, but are not limited to, dimethicone crosspolymer, dimethicone/vinyldimethicone corsspolymer, polysilicone-11 and mixtures thereof. Such elastomers can be used alone or with volatile or nonvolatile solvents Examples of suitable solvents include, but are not limited to, volatile silicones, volatile alcohols, volatile esters, volatile hydrocarbons, and mixtures thereof. The silicone elastomers are crosslinked and preferably have a weight average molecular weight greater than about 100,000. Preferred for use herein are elastomer/solvent blends having an elastomer to solvent ratio of from about 1:100 to about 1:1, more preferably from about 1:30 to about 1:5. Preferably the silicone elastomer blend has a viscosity of from about 50,000 centipoise to about 400,000 centipoise, more preferably from about 100,000 centipoise to about 300, 000 centipoise.

Examples of suitable silicone elastomer blends include cyclomethicone and dimethicone crosspolymer blend (Dow Corning®9040 silicone elastomer); cyclomethicone and dimethicone/vinyldimethicone cross polymer blend (SFE 839 elastomer dispersion available from GE);

octamethylcyclotetrasiloxane and polysilicone-11 blend (Gransil GCM available from Shin Etsu); and mixtures thereof. Preferred herein is the cyclomethicone and dimethicone/vinyldimethicone cross polymer blend.

When present, the silicone elastomer preferably comprises from about 0.01% to about 5%, preferably from about 0.1% to about 2%.

When present, silicone elastomer or gum blends preferably comprise from about 0.1% to about 10%, preferably from about 1% to about 10%, most preferably from about 4% to about 10% by weight of the composition.

ii.) Wax Materials

Wax materials used herein preferably have melting points of at least about or greater than about 20° C., more preferably at least about or greater than about 25° C., and still more preferably at least about or greater than 32° C., and most preferably at least about or greater than about 35° C. The wax materials are preferably soluble in the alcohol antiseptic. The phrase “soluble in the alcohol antiseptic,” as used herein, means the wax materials is soluble in the alcohol antiseptic, at 25° C., at a concentration of 0.1%, preferably 0.2%, more preferably 0.4% by weight, and most preferably soluble at 1.0% by weight. Examples of suitable wax materials include, but are not limited to, dimethicone copolyols having a weight average molecular weight greater than about 1000 such as Biowax®(supplied by Biosil), polyoxyethylene glycols having weight average molecular weight greater than about 500 such as Carbowax (supplied by Union Carbide), and mixtures thereof. Preferred for use herein is Biowax® 754.

Also preferred for use herein are polyoxyethylene glycols having weight average molecular weight greater than about 500, preferably from about 1000 to about 10,000, more preferably from about 1400 to about 6000. Most preferred is PEG-32 (Carbowax 1450).

When present, the above wax materials preferably comprise from about 0.1% to about 10%, preferably from about 0.1% to about 5%, most preferably from about 0.4% to about 2% by weight of the composition.

iii.) Powders

Also useful as degreasing agents are powders. Powder ingredients which may be compounded in the composition of the present invention include inorganic powder such as gums, chalk, Fuller's earth, talc, kaolin, iron oxide, mica, sericite, muscovite, phlogopite, synthetic mica, lepidolite, biotite, lithia mica, vermiculite, magnesium carbonate, calcium carbonate, aluminum silicate, starch, smectite clays, alkyl and/or trialkyl aryl ammonium smectites, chemically modified magnesium aluminum silicate, organically modified montmorillonite clay, hydrated aluminum silicate, fumed silica, aluminum starch octenyl succinate barium silicate, calcium silicate, magnesium silicate, strontium silicate, metal tungstate, magnesium, silica alumina, zeolite, barium sulfate, calcined calcium sulfate (calcined gypsum), calcium phosphate, fluorine apatite, hydroxyapatite, ceramic powder, metallic soap (zinc stearate, magnesium stearate, zinc myristate, calcium palmitate, and aluminum stearate), colloidal silicone dioxide, and boron nitride; organic powder such as polyamide resin powder (nylon powder), cyclodextrin, polyethylene powder, methyl polymethacrylate powder, polystyrene powder, copolymer powder of styrene and acrylic acid, benzoguanamine resin powder, poly(ethylene tetrafluoride) powder, and carboxyvinyl polymer, cellulose powder such as hydroxyethyl cellulose and sodium carboxymethyl cellulose, ethylene glycol monostearate; inorganic white pigments such as titanium dioxide, zinc oxide, and magnesium oxide. Other useful powders are disclosed in U.S. Pat. No. 5,688,831, to El-Nokaly et al., issued Nov. 18, 1997, herein incorporated by reference in its entirety. Preferred for use herein are particulate crosslinked hydrocarbyl-substituted polysiloxane available under the tradename Tospearl from Toshiba Silicone. Mixtures of the above powders may also be used.

Preferably the powders of the present invention have a particle size such that the average chord length of the powder particles range from about 0.01 microns to about 100 microns, preferably from about 0.1 microns to about 50 microns, more preferably from about 1 micron to about 20 microns.

Preferably, the powders of the present invention are spherical or platelet in shape for smooth skin feel. Alternatively and preferably, the powders can by amorphous or irregular shaped for a draggy skin feel. When present, powders preferably comprise from about 0.01% to about 10%, preferably from about 0.1% to about 10%, more preferably from about 0.1% to about 5%, most preferably from about 0.4% to about 2% by weight of the composition.

iv.) Fluorochemicals

Also useful herein are fluorochemicals. These fluorochemicals include fluorotelemers, and perfluoropolyethers, some examples of which are described in Cosmetics & Toiletries, Using Fluorinated Compounds in Topical Preparations, Vol. 111, pages 47-62, (October 1996) which description is incorporated herein by reference. More specific examples of such liquid carriers include, but are not limited to, perfluoropolymethyl isopropyl ethers, perfluoropolypropylethers, acrylamide fluorinated telomer or mixtures thereof. Other more specific examples include, but are not limited to, the polyperfluoroisopropyl ethers available from Dupont Performance Chemicals under the trade name Fluortress® PFPE oils.

When present, powders preferably comprise from about 0.01% to about 10%, preferably from about 0.1% to about 2% by weight of the composition.

Whilst some materials can function either as the lipophilic skin moisturizing agent, thickening agent therefor, or degreasing or detackifying agent, it will be appreciated that the moisturizing, thickening and degreasing or detackifying function cannot be provided by the same component. However, it will be understood that where the composition comprises three or more lipophilic skin moisturizing agents, two of said lipophilic skin moisturizing agents can also function as a thickening agent, or degreasing or detackifying agent.

Stabilizers

When a lipophilic skin moisturizing agent is employed as the mildness enhancer in the compositions herein, a stabilizer may also be included at a level ranging from about 0.1% to about 10%, preferably from about 0.1% to about 8%, more preferably from about 0.1% to about 5% by weight of the antimicrobial composition.

The stabilizer is used to form a crystalline stabilizing network in the liquid cleansing composition that prevents the lipophilic skin moisturizer agent droplets from coalescing and phase splitting in the product. The network exhibits time dependent recovery of viscosity after shearing (e.g., thixotropy).

The stabilizers used herein are not surfactants. The stabilizers provide improved shelf and stress stability. Some preferred hydroxyl-containing stabilizers include 12-hydroxystearic acid, 9,10-dihydroxystearic acid, tri-9,10-dihydroxystearin and tri-12-hydroxystearin (hydrogenated castor oil is mostly tri-12-hydroxystearin). Tri-12-hydroxystearin is most preferred for use in the compositions herein. When these crystalline, hydroxyl-containing stabilizers are utilized in the cleansing compositions herein, they are typically present at from about 0.1% to 10%, preferably from 0.1% to 8%, more preferably from 0.1% to about 5% of the antimicrobial compositions. The stabilizer is insoluble in water under ambient to near ambient conditions.

Alternatively, the stabilizer employed in the cleansing compositions herein can comprise a polymeric thickener. When polymeric thickeners as the stabilizer in the cleansing compositions herein, they are typically included in an amount ranging from about 0.01% to about 5%, preferably from about 0.3% to about 3%, by weight of the composition. The polymeric thickener is preferably an anionic, nonionic, cationic or hydrophobically modifier polymer selected from the group consisting of cationic polysaccharides of the cationic guar gum class with molecular weights of 1,000 to 3,000,000, anionic, cationic, and nonionic homopolymers derived from acrylic and/or methacrylic acid, anionic, cationic, and nonionic cellulose resins, cationic copolymers of dimethyldialkylammonium chloride, and acrylic acid, cationic homopolymers of dimethylalkylammonium chloride, cationic polyalklene and ethoxypolyalkylene imines, polyethylene glycol of molecular weight from 100,000 to 4,000,000, and mixtures thereof. Preferably, the polymer is selected from the group consisting of sodium polyacrylate, hydroxy ethyl cellulose, cetyl hydroxy ethyl Cellulose, and Polyquaternium 10.

Alternatively, the stabilizer employed in the cleansing compositions herein can comprise C10-C22 ethylene glycol fatty acid esters. C10-C22 ethylene glycol fatty acid esters can also desirably be employed in combination with the polymeric thickeners hereinbefore described. The ester is preferably a diester, more preferably a C14-C18 diester, most preferably ethylene glycol distearate. When C10-C22 ethylene glycol fatty acid esters are utilized as the stabilizer in the personal cleansing compositions herein, they are typically present at from about 3% to about 10%, preferably from about 5% to about 8%, more preferably from about 6% to about 8% of the personal cleansing compositions.

Another class of stabilizer which can be employed in the antimicrobial compositions of the present invention comprises dispersed amorphous silica selected from the group consisting of fumed silica and precipitated silica and mixtures thereof. As used herein the term “dispersed amorphous silica” refers to small, finely divided non-crystalline silica having a mean agglomerate particle size of less than about 100 microns.

Fumed silica, which is also known as arced silica, is produced by the vapor phase hydrolysis of silicon tetrachloride in a hydrogen oxygen flame. It is believed that the combustion process creates silicone dioxide molecules which condense to form particles. The particles collide, attach and sinter together. The result of this process is a three dimensional branched chain aggregate. Once the aggregate cools below the fusion point of silica, which is about 1710° C., further collisions result in mechanical entanglement of the chains to form agglomerates. Precipitated silicas and silica gels are generally made in aqueous solution. See, Cabot Technical Data Pamphlet TD-100 entitled “CAB-O-SIL® Untreated Fumed Silica Properties and Functions”, October 1993, and Cabot Technical Data Pamphlet TD-104 entitled “CAB-O-SIL® Fumed Silica in Cosmetic and Personal Care Products”, March 1992, both of which are herein incorporated by reference.

The fumed silica preferably has a mean agglomerate particle size ranging from about 0.1 microns to about 100 microns, preferably from about 1 micron to about 50 microns, and more preferably from about 10 microns to about 30 microns. The agglomerates are composed of aggregates which have a mean particle size ranging from about 0.01 microns to about 15 microns, preferably from about 0.05 microns to about 10 microns, more preferably from about 0.1 microns to about 5 microns and most preferably from about 0.2 microns to about 0.3 microns. The silica preferably has a surface area greater than 50 sq. m/gram, more preferably greater than about 130 sq. m./gram, most preferably greater than about 180 sq. m./gram.

When amorphous silicas are used as the stabilizer herein, they are typically included in the cleansing compositions at levels ranging from about 0.1% to about 10%, preferably from about 0.25% to about 8%, more preferably from about 0.5% to about 5%.

A fourth class of stabilizer which can be employed in the antimicrobial compositions of the present invention comprises dispersed smectite clay selected from the group consisting of bentonite and hectorite and mixtures thereof. Bentonite is a colloidal aluminum clay sulfate. See Merck Index, Eleventh Edition, 1989, entry 1062, p. 164, which is incorporated by reference. Hectorite is a clay containing sodium, magnesium, lithium, silicon, oxygen, hydrogen and flourine. See Merck Index, eleventh Edition, 1989, entry 4538, p. 729, which is herein incorporated by reference.

When smectite clay is employed as the stabilizer in the cleansing compositions of the present invention, it is typically included in amounts ranging from about 0.1% to about 10%, preferably from about 0.25% to about 8%, more preferably from about 0.5% to about 5%.

Other known stabilizers, such as fatty acids and fatty alcohols, can also be employed in the compositions herein. Palmitic acid and lauric acid are especially preferred for use herein.

Other Optional Ingredients

The compositions of the present invention can comprise a wide range of optional ingredients. The CTFA International Cosmetic Ingredient Dictionary, Sixth Edition, 1995, which is incorporated by reference herein in its entirety, describes a wide variety of nonlimiting cosmetic and pharmaceutical ingredients commonly used in the skin care industry, which are suitable for use in the compositions of the present invention. Nonlimiting examples of functional classes of ingredients are described at page 537 of this reference. Examples of these functional classes include: abrasives, anti-acne agents, anticaking agents, antioxidants, binders, biological additives, bulking agents, chelating agents, chemical additives, colorants, cosmetic astringents, cosmetic biocides, denaturants, drug astringents, emulsifiers, external analgesics, film formers, fragrance components, humectants, opacifying agents, plasticizers, preservatives, propellants, reducing agents, skin bleaching agents, skin-conditioning agents (emollient, humectants, miscellaneous, and occlusive), skin protectants, solvents, foam boosters, hydrotropes, solubilizing agents, suspending agents (nonsurfactant), sunscreen agents, ultraviolet light absorbers, and viscosity increasing agents (aqueous and nonaqueous). Examples of other functional classes of materials useful herein that are well known to one of ordinary skill in the art include solubilizing agents, sequestrants, and keratolytics, and the like.

Water-insoluble Substrates

The compositions of the present invention can also be, optionally, incorporated into an insoluble substrate for application to the skin such as in the form of a treated wipe. Suitable water insoluble substrate materials and methods of manufacture are described in Riedel, “Nonwoven Bonding Methods and Materials,” Nonwoven World (1987); The Encyclopedia Americana, vol. 11, pp. 147-153, vol. 21, pp. 376-383, and vol. 26, pp. 566-581 (1984); U.S. Pat. No. 3,485,786 to Evans, issued Dec. 23, 1969;; U.S. Pat. No. 2,862,251, to Kalwarres, issued 1958; U.S. Pat. No. 3,025,585, Kalwarres, issued 1967; U.S. Pat. No. 4,891,227, to Thaman et al., issued Jan. 2, 1990; and U.S. Pat. No. 4,891,228 and U.S. Pat. No. 5,686,088 to Mitra et al., issued Nov. 11,1997; U.S. Pat. No. 5,674,591; James et al; issued Oct. 7, 1997; all of which are herein incorporated by reference in their entirety.

Nonwoven substrates made from synthetic materials useful in the present invention can also be obtained from a wide variety of commercial sources. Nonlimiting examples of suitable nonwoven layer materials useful herein include PGI Miratec Herringbone, a patterned hydroentangled material containing about 30% rayon and 70% polyester, and having a basis weight of about 56 grams per square yard (gsy), available from PGI/Chicopee, Dayton N.J.; PGI Miratec Starburst, a patterned hydroentangled material containing about 30% rayon and 70% polyester, and having a basis weight of about 56 grams per square yard (gsy), available from PGI/Chicopee, Dayton N.J.; Novonet^{R} 149-616, a thermo-bonded grid patterned material containing about 100% polypropylene, and having a basis weight of about 50 gsy, available from Veratec, Inc., Walpole, Mass.; Novonet^{R} 149-801, a thermo-bonded grid patterned material containing about 69% rayon, about 25% polypropylene, and about 6% cotton, and having a basis weight of about 75 gsy, available from Veratec, Inc. Walpole, Mass.; Novonet^{R} 149-191, a thermo-bonded grid patterned material containing about 69% rayon, about 25% polypropylene, and about 6% cotton, and having a basis weight of about 100 gsy, available from Veratec, Inc. Walpole, Mass.; HEF Nubtex^{R} 149-801, a nubbed, apertured hydroentangled material, containing about 100% polyester, and having a basis weight of about 70 gsy, available from Veratec, Inc. Walpole, Mass.; Keybak^{R} 951V, a dry formed apertured material, containing about 75% rayon, about 25% acrylic fibers, and having a basis weight of about 43 gsy, available from PGI/Chicopee, Dayton, N.J.; Keybak^{R} 1368, an apertured material, containing about 75% rayon, about 25% polyester, and having a basis weight of about 39 gsy, available from PGI/Chicopee, Dayton, N.J.; Duralace^{R} 1236, an apertured, hydroentangled material, containing about 100% rayon, and having a basis weight from about 40 gsy to about 115 gsy, available from PGI/Chicopee, Dayton, N.J.; Duralace^{R} 5904, an apertured, hydroentangled material, containing about 100% polyester, and having a basis weight from about 40 gsy to about 115 gsy, available from PGI/Chicopee, Dayton, N.J.; Sontara 8877, an apertured hydroentangled material, containing about 50% Nylon and about 50% Pulp, and having a basis weight of about 68 gsm, available from Dupont Chemical Corp.

Alternatively, the water insoluble substrate can be a polymeric mesh sponge as described in U.S. Pat. No. 5,650,384, incorporated by reference herein in its entirety. The polymeric sponge comprises a plurality of plies of an extruded tubular netting mesh prepared from a strong flexible polymer, such as addition polymers of olefin monomers and polyamides of polycarboxylic acids. Although these polymeric sponges are designed to be used in conjunction with a liquid cleanser, these types of sponges can be used as the water insoluble substrate in the present invention.

Methods for Cleansing and Disinfecting the Skin

The antimicrobial compositions of the present invention are useful for disinfecting and cleansing the skin. Generally, the skin disinfection and cleansing process involves topically applying to the skin a safe and effective amount of a composition of the present invention. The present invention can be used when cleansing processes requiring soap and water are unavailable or inconvenient. The amount of the composition applied, the frequency of application and the period of use will vary widely depending upon the level of disinfection and cleansing desired, e.g., the degree of microbial contamination and/or skin soiling. Preferably the compositions are applied to the skin once daily, more preferably at least three times per day. Typical amounts of antimicrobial composition used preferably range from about 0.1 mg/cm² to about 20 mg/cm², more preferably from about 0.5 mg/cm² to about 10 mg/cm², and most preferably about 1 mg/cm² to about 5 mg/cm² of skin area to be cleansed. Preferably, the antimicrobial compositions of the present invention are used to cleanse and disinfect human and/or animal skin.

The present invention also encompasses the method of applying an effective amount of the antimicrobial compositions of the present invention onto non-skin surfaces, such as household surfaces, e.g., countertops, kitchen surfaces, food preparing surfaces (cutting boards, dishes, pots and pans, and the like); major household appliances, e.g., refrigerators, freezers, washing machines, automatic dryers, ovens, microwave ovens, dishwashers; cabinets; walls; floors; bathroom surfaces, shower curtains; garbage cans and/or recycling bins, and the like.

Article of Manufacture.

The present invention also relates to an article of manufacture comprising a dispensing container containing the antimicrobial composition. Preferably, the container contains instructions for using the antimicrobial compositions of the present invention. Said dispensing container can be constructed of any of the conventional material employed in fabricating containers, including, but not limited to: polyethylene; polypropylene; polyacetal; polycarbonate; polyethyleneterephthalate; polyvinyl chloride; polystyrene; blends of polyethylene, vinyl acetate, and rubber elastomer. Other materials can include stainless steel and glass. A preferred container is made of clear material, e.g., polyethylene terephthalate.

Also preferred is an article of manufacture wherein the dispensing container is a spray dispenser. Said spray dispenser is any of the manually activated means for producing a spray of liquid droplets as is known in. A preferred spray container is made of clear material, e.g., polyethylene terephthalate.

Preparation of the Substrate Material Impregnated with Cleansing Composition

Any method suitable for the application of aqueous or aqueous/alcoholic impregnates, including flood coating, spray coating or metered dosing, can be used to impregnate the fibrous webs herein with the cleansing compositions described herein. More specialized techniques, such as Meyer Rod, floating knife or doctor blade, which are typically used to impregnate liquids into absorbent sheets may also be used.

The emulsion should preferably comprise from about 100% to about 400%, preferably from about 100% to about 300% by weight of the absorbent sheet.

After coating, the sheets may be folded into stacks and packaged in any of the moisture and vapor impermeable packages known in the art.

The anti-microbial cleansing compositions of the present invention are made via art recognized techniques for the various forms compositions.

Methods of Using the Cleansing Wipes

The antimicrobial compositions and wipe of the present invention are useful for personal cleansing, reducing germs on skin, and providing residual effectiveness versus microorganisms such as fungus and bacteria as well as viruses. Typically the wipe is used to apply the cleansing compositions to the area to be cleansed. The wipes herein can be used for personal cleansing when the use of cleansing products requiring water cannot be, or are inconvenient. Typical quantities of the present wipes useful for cleansing, range from about 1 to about 4 wipes per use, preferably from about 1 to about 2 wipes per use. Typical amounts of cleansing composition used range from about 4 mg/cm² to about 6 mg/cm², preferably about 5 mg/cm² of skin area to be cleansed.

### ANALYTICAL TEST METHODS

Residual Anti-viral Efficacy (or Activity) Test

Reference:

1. Sattar, S. A., Standard Test Method for Determining the Virus-Eliminating Effectiveness of Liquid Hygenic Handwash Agents Using the Fingerpads of Adult Volunteers, Annual Book of ASTM Standards. Designation E1838-96, herein incorporated by reference in its entirety and hereinafter referred as “Sattar I”.

2. Sattar, S. A., et al, Chemical Disinfection to Interrupt Transfer of Rhinovirus Type 14 from Environmental Surfaces to Hands, Applied and Environmental Microbiology, Vol. 59, No. 5, May 1993, p.1579-1585, herein incorporated by reference in its entirety and hereinafter referred as “Sattar II”.

The method used to determine the Antiviral index of the present invention is substantially that described by S. A. Satter in Annual Book of ASTM Standards to test the virucidal activity of liquid hand washes (rinse-off products). (See, Sattar I). The method is modified in this case to provide reliable data regarding leave-on products.

Procedure:

Ten Minute Test:

Subjects (5 per test product) initially wash their hands with a non-medicated soap, rinse them and allow them to dry.

The hands are then treated with 70% ethanol and air dried.

Test product (1.0 ml) is applied to the hands, except for the thumbs, and allowed to dry. Test product is applied using a moistened substrate (wipe) and sufficient pressure such that the substrate remains in contact with the skin for a total of 30 seconds.

Approximately 10 minutes (±30 seconds) after product application, 10 μl of a Rhinovirus-14 suspension (ATCC VR-284, approximately 1×10⁸ PFU (plaque forming units)/ml) is topically applied using a micropipette to various sites on the hand a within a designated skin surface area known as fingerpads. At this time, a solution of rhinovirus is also applied to the thumb in a similar manner.

After a dry down period of 7-10 minutes, the virus is then eluted from each of the various skin sites by inverting the mouth of a plastic vial (one vial per site) containing 1 ml of eluent (Minimal Essential media (MEM)+1% pen-strep-glutamate), inverting 20 times per site.

The inoculated skin site is then completely decontaminated by treating the area with a 1:10 dilution of domestic bleach (Clorox® 5.25% Sodium hypochlorite) in tap water, then rinsing with 70% ethanol. Viral titers were determined using standard techniques (plaque assays or TCID₅₀ [Tissue Culture Infectious Dose]).

One Hour Test:

Subjects are allowed to resume normal activities (with the exception of washing their hands) between the 1 hour and 3 hour timepoints. After 1 hour, a rhinovirus suspension was applied to and eluted from designated sites on the fingerpads exactly as described in above for the 10 minute test.

Results:

The TCID₅₀ assay method which is used for measurement of infectious cytocidal virions is described by Burleson, F G, et al; in Virology: A Laboratory Manual, Academic Press, San Diego, Calif., 1992, pp 58-61. Serial dilutions of the eluates from the samples prepared above are added to 96-well plates at 0.1 ml/well. A stock solution of HeLa cells are then pipetted at 0.1 ml/well into each of the wells. All plates are incubated at 33° C. in a CO₂ incubator for three to five days. Plates are monitored microscopically, and cytopathic effects are recorded and calculated using the Reed and Muench calculation of the 50% endpoint as described by Burleson, et. al. Residual anti-viral activity is then calulated by subtracting the Log TCID₅₀ values from treated samples from the log TCID₅₀ values in the control (untreated) samples (defined as log reduction). The average of log reduction values for 5 subjects is reported as the Antiviral Residual Effectiveness Index.

Plaque assay are performed as described by Sattar, S. A., et al, in Chemical Disinfection to Interrupt Transfer of Rhinovirus Type 14 from Environmental Surfaces to Hands, Applied and Environmental Microbiology, Vol. 59, No. 5, May, 1993, p.1579-1585. Confluent HeLa cells are washed once with Earl's Balanced Salt Solution (EBSS), then are treated with serial dilutions of each eluate at 100 μl/well. Plates are placed on rocker table in a 33° C., 5% CO₂ incubator for 1 hour. Unabsorbed virus is aspirated off and an agar overlay (MEM, DEAE-dextran (50 μg/ml), 5-bromo-2′-deoxyuridine (100 μg/ml), 2% fetal bovine serum, and 0.9% Bactoagar) is added at 2 ml/well. Plates are incubated at 33° C., 5% CO₂ for approximately 72 hours. Cells are then fixed and stained, and plaques are counted in each dilution. Residual anti-viral efficacy is then calculated by subtracting the log values of the plaque forming units (PFU) from treated samples from the log PFU values in the control (untreated) samples (defined as log reduction). The average log reduction values for 5 subjects is reported as the Antiviral Residual Effectiveness Index.

In vitro Residual Effectiveness vs. E. coli

Materials

| | |
|---|---|
| Substrate: | Sterile pigskin obtained fresh after slaughter |
| | (defatted, shaved, washed with mild surfactant, and |
| | irradiated to sterilize) |
| Organism: | Eschericia coli ATCC 11229 |
| Suspension broth: | 1/10 Trypticase Soy Broth |
| Culture Suspension: | Overnight culture of the organism in 1/10 TSB, |
| | adjusted in saline, ˜10⁸ CFU/ml (41%-42% |
| | transmittance on the spectrophotometer vs. blank) |
| Agar: | Trypticase Soy Agar + 1.5% Tween 80 |
| Sampling Solution: | 0.04% KH₂ PO₄, 1.01% Na₂HPO₄, 0.11% |
| | Triton-X-100, 1.5% Polysorbate 80, 0.3% Lecithin |
| | Adjust to pH 7.8 |
| Dilution Fluid: | Phosphate Buffered Saline pH 7.2-7.4 (0.117% |
| | Na₂HPO₄, 0.022% NaH₂PO₄, 0.85% NaCl) |

1. Test Design

Residual Antibacterial efficacy of leave-on antimicrobial products are quantified in the following method. Reductions are reported from a no treatment control. By definition the control will show no residual effectiveness in the test.

2. Pre-test Phase

Pigskin is pretreated by submerging 12″×12″ squares of skin into ˜500 ml of wash solution (50:50 v/v Ethanol:Water) and gently agitating the surface with a gloved hand. Each skin is washed 3× with fresh solutions and with a final 500 ml DI water bath in the same manner. Skins are allowed to dry (can be blotted with paper towels), cut into 5 cm² area (˜1″ diameter discs) and frozen until use. Before use, thaw until pigskin reaches room temperature and humidity conditions (˜20 C and X% Relative Humidity).

3. Treatment by Test Product

50 ul of test solution is applied to the pigskin surface, distributed evenly across the surface with an inoculating loop and allowed to dry for 15 minutes. Test solution is the leave-on product being evaluated or lotions expelled from substrate based product after applying pressure.

4. Inoculation Procedure

a) E. coli inoculum (ATCC 10536, grown from lyophilized stock in 1/10 Soybean-casein broth at 37 C for 18-24 hrs) is adjusted to approximately 10⁸ organisms/ml (0.41-0.42 transmittance vs. TSB blank on specrophotometer).

b) Each test site is inoculated with 6.25 μl of E. coli. Inoculum is spread with inoculating loop over the entire 5 cm² area.

c) This procedure is repeated for each test site.

5. Sampling Bacteria (Extraction Procedure)

a) Prepare sampling solution of 0.04% KH₂PO₄, 1.01% Na₂HPO₄, 0.1% Triton X-100, 1.5% Polysorbate 80, 0.3% Lecithin in water, adjusted to pH 7.8 with 1 N HCl.

b) Exactly 10 minutes after inoculation, pigskin disc is placed into a sterile capped 50 mil conical centrifuge tube containing 10 ml of sampling solution.

c) Place tube containing pigskin and sampling solution onto vortex unit and vortex vigorously for 30 seconds.

d) This entire extraction procedure is repeated for each test site 10 minutes after inoculation.

6. Quantifying Bacteria

(any standard quantitative microbiological technique can be used—example is as follows)

a) Prepare phosphate buffer solution of 0.117% Na₂HPO₄, 0.022% NaH₂PO₄, and 0.85% NaCl adjusted to pH 7.2-7.4 with 1 N HCl.

b) 1.1 ml of the sampling solution (immediately after vortexing with pigskin) is aseptically removed from the tube, 0.1 ml of the solution is spread plated onto trypticase-soy agar containing 1.5% Polysorbate 80. Remaining 1 ml is placed into 9 ml of sterile phosphate buffer achieving a 1:10 dilution of the sampling solution. This process is repeated 3 more times (each serial dilution).

c) The plates are inverted and incubated for 24 hours at 35 C.

d) Colonies formed on plates are then enumerated and results are calculated by multiplying the counts by the dilution factor (original sample=10, first dilution=100, second dilution=1000, etc.) and the final results represent the number of colony forming units per ml (CFU's/ml) and are reported as the Log Reduction in bacteria for the sample.

7. Index Calculation

Log Reduction=log₁₀(CFU's/ml of placebo site)−log₁₀(CFU's/ml of test product site)

The average of the Log Reduction values of the six samples is reported as the Antibacterial (or Gram Negative) Residual Efficacy Index.

Forearm Controlled Application Test (FCAT)

Reference:

Ertel, K. D., et al.; “A Forearm Controlled Application Technique for Estimating the Relative Mildness of Personal Cleansing Products”; J. Soc. Cosmet. Chem. 46 (1995) 67-76

The Forearm Controlled Application Test, or FCAT, is a comparative test which discriminates differences in product mildness to the skin. A test product is compared to a standard soap based cleansing bar control.

Test Group Restrictions

Test groups of 20-30 subjects, 18 to 55 years of age, who regularly wash with soap are used. Potential subjects who (1) have an initial dryness grade of 3.0 or higher on the forearms as assessed during the initial examination, (2) have skin cancer, eczema, or psoriasis on the forearms, (3) are receiving injectable insulin, (4) are pregnant or lactating, or (5) are receiving treatment for skin problems or contact allergy are excluded. Subjects are to avoid hot tubs, swimming, and sun lamps, and to refrain from applying any soaps, cleansing products, creams, or gels to their forearms for the duration of the study. Subjects are to keep water off their forearms for at least two hours before the grading process. The studies are executed using a blinded, random product order format. Clinical assistant should verify the correct treatment sequence and document such before washing each subject.

Products are applied to the forearms a total of nine (9) times: two (2) times each day on the first four (4) days of the study and one (1) time on the final day. Visits to the test facility for washing must be spaced by a minimum of three (3) hours.

All clinical assistants must wear disposable gloves during wash procedure, rinsing them between treatments, and changing between subjects.

Control Product

The control product is a rolled bar soap containing:

| | |
|---|---|
| 56.1% | Sodium Tallowate |
| 18.7% | Sodium Cocoate |
| 0.7% | Sodium Chloride |
| 24% | Water |
| 0.5% | Minors (Perfume, Impurities) |

Test Product

Test products are prepared by incorporating the desired test materials using conventional mixing technologies. These products can range from simple solutions (e.g., acid in water) to complex product formulations.

Product Application Procedure

Both test and control products are tested on the same arm. The following test procedure is used.

1. The subject wets the entire surface of his/her volar forearm with 95-100° F. tap water by holding the arm briefly under running tap water.

2. A clinical assistant wets one-quarter sheet (approximately 8″×6″) of Masslinn® towel with tap water, then squeezes the towel gently to remove excess water.

3. A clinical assistant applies the products to the arm, beginning with the product designated for the site nearest the elbow, using the appropriate procedure as follows:

Liquid Product

a. Dispense 0.10 cc of test product from a syringe into the center of the appropriate marked area.

b. Wet two finders of gloved (latex) hand under the running tap (index and middle fingers).

c. Move wetted fingers in a circular motion over the application site for 10 seconds to lather product.

d. Lather remains on the application site for 90 seconds, then is rinsed off with running tap water for 15 seconds, taking care not to wash lather off the adjacent sites. After 10 seconds of the rinse has expired, the Clinical Assistant will gently rub the site being rinsed with her two gloved fingers for the remaining 5 seconds of the rinse.

Bar Product

a. Wet two finders of gloved (latex) hand under the running tap (index and middle fingers).

b. Wet bar by holding bar briefly under running tap water. Test bars must be wet under a running tap at the start of each day.

c. Rub wetted fingers in a circular motion, over the surface of the bar, for 15 seconds to form lather on bar and fingers.

d. Rub the lathered fingers on the application site in a circular motion for 10 seconds to lather product on the skin.

e. Lather remains on the application site for 90 seconds, then is rinsed off with running tap water for 15 seconds, taking care not to wash lather off the adjacent sites. After 10 seconds of the rinse has expired, the Clinical Assistant will gently rub the site being rinsed with her two gloved fingers for the remaining 5 seconds of the rinse.

Wipe Products

a. Fold wipe in half, crosswise, and gently rub the wipe in a curricular motion within the appropriate area.

b. Allow site to air dry for 90 seconds. Do not rinse site.

Leave-on Product

a. Dispense 0.10 cc of test product from a syringe into the center of the appropriate marked area.

b. Move gloved fingers in a circular motion over the application site for 10 seconds.

c. Allow site to air dry for 90 seconds. Do not rinse site.

4. While waiting for the 90 second residence time to expire, the above procedure will be repeated on the remaining application site on that arm, working down the arm toward the wrist.

5. Steps 1-4 are repeated on the appropriate test areas so two applications of product are made to test areas.

6. After all of the application areas have two applications of products, the clinical assistant gently pats the subjects arm dry with a disposable paper towel.

Evaluation

The skin on each treatment area is evaluated by an expert grader at baseline and three hours after the final study wash. The treatment areas are evaluated under 2.75× magnification (model KFM-1A Luxo Illuminated Magnifying Lamp, Marshall Industries, Dayton, Ohio) with controlled lighting (General Electric Cool White, 22-watt, 8″ Circuline fluorescent bulb).

The skin is evaluated by an expert grader ,for dryness and a rating is assigned based on the definitions set forth below.

TABLE 1
Forearm Grading Scale

| Rating | Skin Dryness |
|---|---|
| 0 | No dryness |
| 1.0 | Patches of slight powderiness and occasional patches of small |
| | scales may be seen. |
| 2.0 | Generalized slight powderiness. Early cracking or occasional |
| | small lifting scales may be present. |
| 3.0 | Generalized moderate powderiness and/or heavy cracking and |
| | lifting scales. |
| 4.0 | Generalized heavy powderiness and/or heavy cracking and |
| | lifting scales. |
| 5.0 | Generalized high cracking and lifting scales. Eczematous change |
| | may be present. Powderiness may be present but not prominent. |
| | May see bleeding crack. |
| 6.0 | Generalized severe cracking. Eczematous change may be |
| | present. Bleeding cracks may be present. Scales large, may be |
| | beginning to disappear. |

The FCAT generally produces only mild to moderate skin irritation; however, if a treated site reaches a rating of 5.0 or greater, at any time during the study, treatment of all sites on that subject should be discontinued.

Data

After all subjects have been evaluated at the end of the test, the following values are determined:

Rcₒ=The average rating of control product area at baseline

Rc_{f}=The average rating of control product area at test end

Rtₒ=The average rating of test product area at baseline

Rt_{f}=The average rating if test product area at test end.

There are many external conditions which could influence the FCAT, such as relative humidity and water softness. The test is valid only if sufficient response is observed in the skin to the control product. The control response must be greater than 1.0 (i.e., Rc_{f}−Rcₒ≧1.0) for the test to be valid.

Given a valid test, the Mildness Index of the test product is the difference in the skin responses to two products.

Mildness Index=(Rc_{f}−Rcₒ)−(Rt_{f}−Rtₒ)

Sting Test Method

The objective was to compare the level of stinging and/or burning produced by a test material versus a control after a single application to the cheek. Subjects are first screened for their ability to experience a stinging/burning sensation in response to 4% citric acid in comparison to water (control).

The screening process evolves:

i.) describing the psychophysical attribute of sting and the relative intensity of various types of “stinging” sensations (i.e., bee sting, paper cut sensation, application of vinegar to a cut, a splash of alcohol on shaven face, etc.).

ii. ) shaving the facial cheeks of the using Gillette Good News Disposable Razors and Barbasol Non-Medicated Shaving Cream.

iii.) rinsing the cheeks with running tap water (95-100 F) to remove residual shaving cream.

iv.) applying to the cheeks (10 rotations using moderate pressure) 1 ml of product (water or citric acid) and then “splash” rinsing the product off the cheeks after 5 seconds. For a initial evaluation and in order to properly identify sting, subjects are told the products they are to evaluate. The evaluation is then repeated using the second product (i.e., water or citric acid product not initially test). After 48 hours, subjects are qualified based on their ability to distinguish between 4% citric acid vs. water, by at least 2 grades (i.e., a score ≧±2). The “grades” are determined using the following scale:

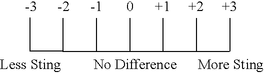

Once qualified, subjects evaluate “test” products as described above, except each product now evaluated independently for sting. The product is lathered with 10 full hand circles (10 sec.), applied to the face, rinsed off, and sting evaluated. Subjects record the intensity of sting using a scale of zero to 8, where zero represents no sting and 8 representing very much or maximum sting intensity. The results are reported as the Sting Index.

### EXAMPLES

The following examples further describe and demonstrate embodiments within the scope of the present invention. In the following examples, all ingredients are listed at an active level. The examples are given solely for the purpose of illustration and are not to be construed as limitations of the present invention, as many variations thereof are possible without departing from the spirit and scope of the invention.

Ingredients are identified by chemical or CTFA name.

### Example 1

The following is an example of a water-insoluble substrate useful in the present invention.

A patterned hydroentangled non-woven substrate having a basis weight of 56 gsy, comprising 70% polyester and 30% rayon approximately 6.5 inches wide by 7.5 inches long with a caliper of about 0.80 mm. Optionally, the substrate can be pre-coated with dimethicone (Dow Corning 200 Fluid 5cst) using conventional substrate coating techniques.

### Examples 2-7

The following are examples of aqueous antimicrobial lotions of the present invention. The compositions are formed by combining and mixing the ingredients of each column using conventional technology and then applying an appropriate amount of the composition to the skin.

| | Exam- | Exam- | Exam- | Exam- | Exam- | Exam- |
|---|---|---|---|---|---|---|
| | ple 2 | ple 3 | ple 4 | ple 5 | ple 6 | ple 7 |
| | Weight | Weight | Weight | Weight | Weight | Weight |
| Ingredient | % | % | % | % | % | % |
| Pyrrolidone | 4 | 4 | 5 | 4 | — | — |
| Carboxylic | | | | | | |
| Acid | | | | | | |
| Sodium PCA | — | — | — | — | 4.68 | 4.68 |
| Ammonium | — | — | 2 | — | — | — |
| Lauryl Sulfate | | | | | | |
| Cocamine | 0.5 | 0.5 | — | 0.25 | 0.25 | — |
| Oxide | | | | | | |
| Lauramine | — | — | — | — | — | 0.38 |
| Oxide | | | | | | |
| Benzalkonium | 0.1 | 0.1 | 0.1 | 0.1 | 0.1 | 0.1 |
| Chloride | | | | | | |
| Tospearl 2000 | 2 | 2 | — | — | — | — |
| Microsilk 419 | — | — | — | — | — | 1 |
| Ethanol | 10 | 10 | 10 | — | — | — |
| Dow Corning | 0.03 | 0.03 | — | 0.03 | 0.03 | 0.1 |
| Antifoam 1510 | | | | | | |
| Sodium | 0.2 | 0.2 | 0.2 | 0.2 | 0.2 | 0.2 |
| Benzoate | | | | | | |
| Tetrasodium | 0.1 | 0.1 | 0.1 | 0.1 | 0.1 | 0.1 |
| EDTA | | | | | | |
| Sodium | 0.4 | 0.4 | 0.4 | 0.4 | — | — |
| Chloride | | | | | | |
| Perfume | 0.01 | 0.01 | 0.01 | 0.01 | 0.01 | 0.01 |
| Sodium | to pH = | to pH = | to pH = | to pH = | to pH = | to pH = |
| Hydroxide/ | 3.0 | 4.0 | 3.0 | 3.0 | 3.0 | 3.0 |
| Hydrochloric | | | | | | |
| Acid | | | | | | |
| Water | Q.S. | Q.S. | Q.S. | Q.S. | Q.S. | Q.S. |
| Mildness Index | >1.6 | >1.6 | >1.6 | >1.6 | >1.6 | >1.6 |
| Antibacterial | >2 | >2 | >2 | >2 | >2 | >2 |
| Residual Effec- | | | | | | |
| tiveness Index | | | | | | |
| Ten Min Anti- | >2 | >2 | >2 | >2 | >2 | >2 |
| viral Residual | | | | | | |
| Effectiveness | | | | | | |
| Index | | | | | | |
| One Hour Anti- | >1 | >1 | >1 | >1 | >1 | >1 |
| viral Residual | | | | | | |
| Effectiveness | | | | | | |
| Index | | | | | | |
| Acid Sting | <3 | <3 | <3 | <3 | <3 | <3 |
| Index | | | | | | |

Alternatively, the above described aqueous antimicrobial lotions can be applied onto the substrate of Example 1 at a lotion to wipe weight ratio of about 2:1 using conventional substrate coating techniques for application to the skin as an antimicrobial and cleansing wipe.

### Example 8

The following is an example of a sanitizing spray composition. The compositions are formed by combining and mixing the ingredients of each column using conventional technology, transferring the composition into a spray bottle, and then spraying an appropriate amount of the composition on the skin.

| Ingredient | Weight % |
|---|---|
| Pyrrolidone Carboxylic Acid | 4 |
| Lauramine Oxide | 0.38 |
| Ethanol | 55 |
| Perfume | 0.05 |
| Sodium Hydroxide/ | to pH = 3.0 |
| Hydrochloric Acid | |
| Water | Q.S. |
| Mildness Index | >1.6 |
| Antibacterial Residual | >2 |
| Effectiveness Index | |
| Ten Min Antiviral Residual | >2 |
| Effectiveness Index | |
| One Hour Antiviral Residual | >1 |
| Effectiveness Index | |
| Acid Sting Index | <3 |

What is claimed is:

1. A leave-on antimicrobial composition comprising;
a.) a proton donating agent; and
b.) a surfactant
wherein the composition has:
i.) a Mildness Index of greater than about 0.3;
ii.) an Antibacterial Residual Effectiveness Index of greater than about 1.0; and
iii.) a Ten Minute Antiviral Residual Effectiveness Index of greater than about 1.0.
and wherein the pH of the composition is less than 5 and wherein the proton donating agent has a sting index of less than 3.5 and is substantially free of salicylic acid.

2. An antimicrobial composition according to claim 1, wherein the Mildness Index of greater than about 0.6.

3. An antimicrobial composition according to claim 2, wherein the Mildness Index of greater than about 1.0.

4. An antimicrobial composition according to claim 3, wherein the Mildness Index of greater than about 1.6.

5. An antimicrobial composition according to claim 1, wherein the Antibacterial Residual Effectiveness Index of greater than about 1.5.

6. An antimicrobial composition according to claim 5, wherein the Antibacterial Residual Effectiveness Index of greater than about 2.0.

7. An antimicrobial composition according to claim 6, wherein the Antibacterial Residual Effectiveness Index of greater than about 2.5.

8. An antimicrobial composition according to claim 1, wherein the Ten Minute Antiviral Residual Effectiveness Index of greater than about 1.5.

9. An antimicrobial composition according to claim 8, wherein the Ten Minute Antiviral Residual Effectiveness Index of greater than about 2.0.

10. An antimicrobial composition according to claim 9, wherein the Ten Minute Antiviral Residual Effectiveness Index of greater than about 2.5.

11. An antimicrobial composition according to claim 1, wherein the One Hour Antiviral Residual Effectiveness Index of greater than about 1.0.

12. An antimicrobial composition according to claim 11, wherein the One Hour Antiviral Residual Effectiveness Index of greater than about 2.0.

13. An antimicrobial composition according to claim 12, wherein the One Hour Antiviral Residual Effectiveness Index of greater than about 2.5.

14. An antimicrobial composition according to claim 1, wherein the buffering capacity of the acid is greater than 0.005%.

15. An antimicrobial composition according to claim 14, wherein the buffering capacity of the acid is greater than 0.01%.

16. An antimicrobial composition according to claim 15, wherein the buffering capacity of the acid is greater than 0.02%.

17. An antimicrobial composition according to claim 16, wherein the buffering capacity of the acid is greater than 0.04%.

18. An antimicrobial composition according to claim 1, wherein the surfactant is selected from the group consisting of anionic surfactants, cationics, amphoteric or zwitterionic surfactants, and mixturess thereof.

19. An antimicrobial composition according to claim 18, wherein the surfactant contains at least one branched or unbranched, saturated or unsaturated alkyl chain of from 8 to 24 carbon atoms.

20. An antimicrobial composition according to claim 19, wherein the surfactant contains at least one branched or unbranched, saturated or unsaturated alkyl chain of from 12 to 16 carbon atoms.

21. An antimicrobial composition according to claim 20, wherein the surfactant is selected from alkyl sulfates; alkyl ether sulfates; alkyl benzene sulfonates, alpha olefin sulfonates; primary or secondary alkyl sulfonates, alkyl dimethyl amine oxides, alkyl betaines and mixtures thereof.

22. An antimicrobial composition according to claim 18, wherein the surfactant is alkoxylated and wherein the degree of alkoxylation ranges from 1 to 10 alkoxy units.

23. An antimicrobial composition according to claim 1, wherein the proton donating agent has a Sting Index of less than 3.0.

24. An antimicrobial composition according to claim 23, wherein the proton donating agent has a Sting Index of less than 2.5.

25. An antimicrobial composition according to claim 24, wherein the proton donating agent has a Sting Index of less than 2.0.

26. An antimicrobial composition according to claim 1, wherein the proton donating agent is is selected from the group consisting of mono- or di-carboxylic acids, salts thereof and mixtures thereof.

27. An antimicrobial composition according to claim 1 in the form of a sanitizer, spray, foam, gel, cream, lotion, powder, ointment, tincture.

28. An antimicrobial cleansing wipe, comprising:
A. one or more layers of water-insoluble substrate; and
B. a safe and effective amount of leave-on antimicrobial composition comprising;
a) a proton donating agent; and
b) a surfactant
wherein the composition has:
i. a Mildness Index of greater than about 0.3;
ii. an Antibacterial Residual Effectiveness Index of greater than about 1.0; and
iii. a Ten Minute Antiviral Residual Effectiveness Index of greater than about 1.0
and wherein the pH of the composition is less than 5 and wherein the proton donating agent has a sting index of less than 3.5 and the composition is substantially free of salicylic acid.

29. An antimicrobial composition according to claim 28, wherein the Mildness Index of greater than about 0.6.

30. An antimicrobial composition according to claim 28, wherein the Antibacterial Residual Effectiveness Index of greater than about 1.5.

31. An antimicrobial composition according to claim 28, wherein the Ten Minute Antiviral Residual Effectiveness Index of greater than about 1.5.

32. An antimicrobial composition according to claim 28, wherein the One Hour Antiviral Residual Effectiveness Index of greater than about 1.0.

33. An antimicrobial composition according to claim 28, wherein the buffering capacity of the acid is greater than 0.005%.

34. A method for cleansing and disinfecting skin comprising the applying a safe and effective amount of the composition of claim 1 on mammalian skin.

35. A method for cleansing and disinfecting skin comprising the step of applying the antimicrobial cleansing wipe of claim 28 on mammalian skin.

36. A method for skin conditions caused by microbial infection comprising the applying a safe and effective amount of the composition of claim 1 on mammalian skin.

37. A method for skin conditions caused by microbial infection comprising the step of applying the antimicrobial cleansing wipe of claim 28 on mammalian skin.

38. A method for treating acne comprising the use of a safe and effective amount of the composition of claim 1 on human skin.

39. A method of providing immediate inactivation or destruction of a susceptible virus comprising the step of contacting the virus with a safe and effective amount of a composition comprising a proton donating agent, wherein the Mildness Index of the proton donating agent is greater than about 1.6.

40. A method of providing immediate inactivation or destruction of a susceptible virus comprising the step of contacting the virus with a safe and effective amount of a composition comprising a proton donating agent, wherein the Sting Index of the proton donating agent is less than about 3.5.

41. A method of inactivating or destroying a susceptible virus comprising the step of contacting the virus with a safe and effective amount of a composition comprising:
a) a proton donating agent; and
a) a surfactant
wherein the composition has:
i.) a Mildness Index of greater than about 0.3;
ii.) an Antibacterial Residual Effectiveness Index of greater than about 1.0; and
iii.) a Ten Minute Antiviral Residual Effectiveness Index of greater than about 1.0
and wherein the pH of the composition is less than 5 wherein the proton donating agent has a sting index of less than 3.5.

42. A leave-on antimicrobial composition comprising;
a) a proton donating agent; and
b) a surfactant
wherein the proton donating agent and surfactant combination has:
i.) a Mildness Index of greater than about 0.3;
ii.) an Antibacterial Residual Effectiveness Index of greater than about 1.0; and
iii.) a Ten Minute Antiviral Residual Effectiveness Index of greater than about 1.0
and wherein the pH of the composition is less than 5 and wherein the proton donating agent has a sting index of less than 3.5 and the composition is substantially free of salicylic acid.

43. An article of manufacture, comprising a container containing a leave-on antimicrobial composition comprising:
a) a proton donating agent; and
b) a surfactant
wherein the proton donating agent and surfactant combination has:
i.) a Mildness Index of greater than about 0.3;
ii.) an Antibacterial Residual Effectiveness Index of greater than about 1.0; and
iii.) a Ten Minute Antiviral Residual Effectiveness Index of greater than about 1.0
and wherein the pH of the composition is less than 5 and wherein said container has instructions for inactivating and/or destroying viruses on the skin during the cleansing process, said instructions comprising instruction to use the composition on skin at least once daily.

44. An article of manufacture according to claim 43, wherein the proton donating agent has a sting index of less than 3.5

45. An article of manufacture according to claim 43, comprising instruction to use the composition on skin at least 3 times per day.
